SEQUENCE LISTING (103 sequences)

SEQ ID NO: 1 - DNA, 2283 bp, synthetic construct
  source 1..2283: mol_type = other DNA
atgagccctaagaaggtgcagatcaaggtggaagaaaaggaggacgagacagaagagagctcttctgaagaggaagaggaagtggaagataagctgccacggagagaaagcctgcgccccaagagaaagagaaccagagatgtgatcaacgaggacgatcctgaacctgagcctgaggacgaggaaacaagaaaagcacgagagaaggaacggcggagaaggctgaagcggggcgctgaggaagaagaaatcgacgaggaggagctggaaagacttaaggccgagctggatgagaagcgccaaatcatcgccacagtgaaatgcaagccctggaagatggaaaagaaaatcgaggtgctgaaggaggccaaaaagttcgtctccgagaatgagggcgccctcggtaagggcaagggcaagcggtggttcgccttcaagatgatgatggctaagaagtgggccaagttcctgcgggacttcgagaatttcaaggccgcttgtgtgccttgggaaaacaagattaaggcaatcgagagccaatttggaagcagcgtggcctcctactttctgtttctgagatggatgtacggagtgaacatggtgctgttcatcctgacatttagcctgatcatgctgcctgagtacctatggggcctgccctacggcagcctgcctcggaagaccgtgcctcgggccgaagaggccagcgccgccaacttcggcgtgctgtacgacttcaacggccttgctcagtacagcgtgctgttttacggctactacgacaacaaacggaccatcggctggatgaacttcagactgcctctgagctacttcctggtgggcatcatgtgcatcggatactctttcctggttgtgctcaaagccatgaccaagaacatcggcgacgacggcggaggcgatgataacaccttcaatttttcttggaaagtgttcaccagctgggactacctgattggaaaccctgagacagccgataataagttcaatagcatcaccatgaacttcaaggaagccatcaccgaggagaaagccgcccaagtggaagaaaacgttcatctgatcagattcctgagattcctggccaactttttcgtgttcctgaccctgggcggaagcggctacctgatcttctgggccgtgaagcggagccaggagttcgcccagcaggaccccgacaccctgggatggtgggaaaagaacgagatgaacatggtgatgtctctgctcggcatgttctgtcctaccctgttcgacctgtttgccgagctggaggattaccaccctctgatcgccctgaagtggctgctgggtagaatcttcgccctgctgctcggcaacctgtatgtgttcatcctggccctgatggatgagatcaacaataagatcgaagaggaaaagctggtgaaggccaacattaccctgtgggaagccaatatgatcaaggcctataacgcctccttcagcgaaaacagcaccggccctccattcttcgtgcaccccgccgacgtgccaagaggcccttgctgggagacaatggtaggccaggagtttgttagactcacagtgtccgatgtgctgaccacatacgtgaccatcctgatcggcgacttcctgagggcctgcttcgtcagattttgcaactactgctggtgctgggacctggagtacggctaccccagctatacagagttcgacatcagcggcaacgtcctggccctgatctttaaccagggcatgatttggatgggcagcttcttcgcccctagcctgcctggcatcaacatcctgagactgcacacaagcatgtacttccagtgctgggccgtgatgtgttgtaacgtgcccgaggctagagtgttcaaggccagcagaagcaacaacttctacctgggcatgctgcttctgatcctgttcctgtccaccatgcctgtgctgtacatgatcgtgtccctgccacctagcttcgactgcggccctttcagtggaaaaaacagaatgttcgaggtgatcggcgagaccctggagcacgactttccctcttggatggctaagatcctgcggcagctgtctaatcctggcctcgtgatcgccgtgatcctggtcatggtgctggctatctactacctgaacgccaccgctaagggacagaaggccgccaacctggacctgaaaaagaagatgaaaatgcaggccctggaaaacaagatgagaaacaagaagatggccgccgctagagccgctgccgctgctggcagacagtga

SEQ ID NO: 2 - DNA, 260 bp, synthetic construct
  source 1..260: mol_type = other DNA
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 3 - DNA, 56 bp, synthetic construct
  source 1..56: mol_type = other DNA
aataaaggaaatttattttcattgcaatagtgtgttggaattttttgtgtctctca

SEQ ID NO: 4 - skipped

SEQ ID NO: 5 - DNA, 542 bp, synthetic construct
  source 1..542: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 6 - DNA, 130 bp, synthetic construct
  source 1..130: mol_type = other DNA
ctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 7 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 4125 bp, synthetic construct
  source 1..4125: mol_type = other DNA
ctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcccgcggatgagccctaagaaggtgcagatcaaggtggaagaaaaggaggacgagacagaagagagctcttctgaagaggaagaggaagtggaagataagctgccacggagagaaagcctgcgccccaagagaaagagaaccagagatgtgatcaacgaggacgatcctgaacctgagcctgaggacgaggaaacaagaaaagcacgagagaaggaacggcggagaaggctgaagcggggcgctgaggaagaagaaatcgacgaggaggagctggaaagacttaaggccgagctggatgagaagcgccaaatcatcgccacagtgaaatgcaagccctggaagatggaaaagaaaatcgaggtgctgaaggaggccaaaaagttcgtctccgagaatgagggcgccctcggtaagggcaagggcaagcggtggttcgccttcaagatgatgatggctaagaagtgggccaagttcctgcgggacttcgagaatttcaaggccgcttgtgtgccttgggaaaacaagattaaggcaatcgagagccaatttggaagcagcgtggcctcctactttctgtttctgagatggatgtacggagtgaacatggtgctgttcatcctgacatttagcctgatcatgctgcctgagtacctatggggcctgccctacggcagcctgcctcggaagaccgtgcctcgggccgaagaggccagcgccgccaacttcggcgtgctgtacgacttcaacggccttgctcagtacagcgtgctgttttacggctactacgacaacaaacggaccatcggctggatgaacttcagactgcctctgagctacttcctggtgggcatcatgtgcatcggatactctttcctggttgtgctcaaagccatgaccaagaacatcggcgacgacggcggaggcgatgataacaccttcaatttttcttggaaagtgttcaccagctgggactacctgattggaaaccctgagacagccgataataagttcaatagcatcaccatgaacttcaaggaagccatcaccgaggagaaagccgcccaagtggaagaaaacgttcatctgatcagattcctgagattcctggccaactttttcgtgttcctgaccctgggcggaagcggctacctgatcttctgggccgtgaagcggagccaggagttcgcccagcaggaccccgacaccctgggatggtgggaaaagaacgagatgaacatggtgatgtctctgctcggcatgttctgtcctaccctgttcgacctgtttgccgagctggaggattaccaccctctgatcgccctgaagtggctgctgggtagaatcttcgccctgctgctcggcaacctgtatgtgttcatcctggccctgatggatgagatcaacaataagatcgaagaggaaaagctggtgaaggccaacattaccctgtgggaagccaatatgatcaaggcctataacgcctccttcagcgaaaacagcaccggccctccattcttcgtgcaccccgccgacgtgccaagaggcccttgctgggagacaatggtaggccaggagtttgttagactcacagtgtccgatgtgctgaccacatacgtgaccatcctgatcggcgacttcctgagggcctgcttcgtcagattttgcaactactgctggtgctgggacctggagtacggctaccccagctatacagagttcgacatcagcggcaacgtcctggccctgatctttaaccagggcatgatttggatgggcagcttcttcgcccctagcctgcctggcatcaacatcctgagactgcacacaagcatgtacttccagtgctgggccgtgatgtgttgtaacgtgcccgaggctagagtgttcaaggccagcagaagcaacaacttctacctgggcatgctgcttctgatcctgttcctgtccaccatgcctgtgctgtacatgatcgtgtccctgccacctagcttcgactgcggccctttcagtggaaaaaacagaatgttcgaggtgatcggcgagaccctggagcacgactttccctcttggatggctaagatcctgcggcagctgtctaatcctggcctcgtgatcgccgtgatcctggtcatggtgctggctatctactacctgaacgccaccgctaagggacagaaggccgccaacctggacctgaaaaagaagatgaaaatgcaggccctggaaaacaagatgagaaacaagaagatggccgccgctagagccgctgccgctgctggcagacagtgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 3577 bp, synthetic construct
  source 1..3577: mol_type = other DNA
ctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcccgcggatgagccctaagaaggtgcagatcaaggtggaagaaaaggaggacgagacagaagagagctcttctgaagaggaagaggaagtggaagataagctgccacggagagaaagcctgcgccccaagagaaagagaaccagagatgtgatcaacgaggacgatcctgaacctgagcctgaggacgaggaaacaagaaaagcacgagagaaggaacggcggagaaggctgaagcggggcgctgaggaagaagaaatcgacgaggaggagctggaaagacttaaggccgagctggatgagaagcgccaaatcatcgccacagtgaaatgcaagccctggaagatggaaaagaaaatcgaggtgctgaaggaggccaaaaagttcgtctccgagaatgagggcgccctcggtaagggcaagggcaagcggtggttcgccttcaagatgatgatggctaagaagtgggccaagttcctgcgggacttcgagaatttcaaggccgcttgtgtgccttgggaaaacaagattaaggcaatcgagagccaatttggaagcagcgtggcctcctactttctgtttctgagatggatgtacggagtgaacatggtgctgttcatcctgacatttagcctgatcatgctgcctgagtacctatggggcctgccctacggcagcctgcctcggaagaccgtgcctcgggccgaagaggccagcgccgccaacttcggcgtgctgtacgacttcaacggccttgctcagtacagcgtgctgttttacggctactacgacaacaaacggaccatcggctggatgaacttcagactgcctctgagctacttcctggtgggcatcatgtgcatcggatactctttcctggttgtgctcaaagccatgaccaagaacatcggcgacgacggcggaggcgatgataacaccttcaatttttcttggaaagtgttcaccagctgggactacctgattggaaaccctgagacagccgataataagttcaatagcatcaccatgaacttcaaggaagccatcaccgaggagaaagccgcccaagtggaagaaaacgttcatctgatcagattcctgagattcctggccaactttttcgtgttcctgaccctgggcggaagcggctacctgatcttctgggccgtgaagcggagccaggagttcgcccagcaggaccccgacaccctgggatggtgggaaaagaacgagatgaacatggtgatgtctctgctcggcatgttctgtcctaccctgttcgacctgtttgccgagctggaggattaccaccctctgatcgccctgaagtggctgctgggtagaatcttcgccctgctgctcggcaacctgtatgtgttcatcctggccctgatggatgagatcaacaataagatcgaagaggaaaagctggtgaaggccaacattaccctgtgggaagccaatatgatcaaggcctataacgcctccttcagcgaaaacagcaccggccctccattcttcgtgcaccccgccgacgtgccaagaggcccttgctgggagacaatggtaggccaggagtttgttagactcacagtgtccgatgtgctgaccacatacgtgaccatcctgatcggcgacttcctgagggcctgcttcgtcagattttgcaactactgctggtgctgggacctggagtacggctaccccagctatacagagttcgacatcagcggcaacgtcctggccctgatctttaaccagggcatgatttggatgggcagcttcttcgcccctagcctgcctggcatcaacatcctgagactgcacacaagcatgtacttccagtgctgggccgtgatgtgttgtaacgtgcccgaggctagagtgttcaaggccagcagaagcaacaacttctacctgggcatgctgcttctgatcctgttcctgtccaccatgcctgtgctgtacatgatcgtgtccctgccacctagcttcgactgcggccctttcagtggaaaaaacagaatgttcgaggtgatcggcgagaccctggagcacgactttccctcttggatggctaagatcctgcggcagctgtctaatcctggcctcgtgatcgccgtgatcctggtcatggtgctggctatctactacctgaacgccaccgctaagggacagaaggccgccaacctggacctgaaaaagaagatgaaaatgcaggccctggaaaacaagatgagaaacaagaagatggccgccgctagagccgctgccgctgctggcagacagtgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 4223 bp, synthetic construct
  source 1..4223: mol_type = other DNA
ctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcggatgagccctaagaaggtgcagatcaaggtggaagaaaaggaggacgagacagaagagagctcttctgaagaggaagaggaagtggaagataagctgccacggagagaaagcctgcgccccaagagaaagagaaccagagatgtgatcaacgaggacgatcctgaacctgagcctgaggacgaggaaacaagaaaagcacgagagaaggaacggcggagaaggctgaagcggggcgctgaggaagaagaaatcgacgaggaggagctggaaagacttaaggccgagctggatgagaagcgccaaatcatcgccacagtgaaatgcaagccctggaagatggaaaagaaaatcgaggtgctgaaggaggccaaaaagttcgtctccgagaatgagggcgccctcggtaagggcaagggcaagcggtggttcgccttcaagatgatgatggctaagaagtgggccaagttcctgcgggacttcgagaatttcaaggccgcttgtgtgccttgggaaaacaagattaaggcaatcgagagccaatttggaagcagcgtggcctcctactttctgtttctgagatggatgtacggagtgaacatggtgctgttcatcctgacatttagcctgatcatgctgcctgagtacctatggggcctgccctacggcagcctgcctcggaagaccgtgcctcgggccgaagaggccagcgccgccaacttcggcgtgctgtacgacttcaacggccttgctcagtacagcgtgctgttttacggctactacgacaacaaacggaccatcggctggatgaacttcagactgcctctgagctacttcctggtgggcatcatgtgcatcggatactctttcctggttgtgctcaaagccatgaccaagaacatcggcgacgacggcggaggcgatgataacaccttcaatttttcttggaaagtgttcaccagctgggactacctgattggaaaccctgagacagccgataataagttcaatagcatcaccatgaacttcaaggaagccatcaccgaggagaaagccgcccaagtggaagaaaacgttcatctgatcagattcctgagattcctggccaactttttcgtgttcctgaccctgggcggaagcggctacctgatcttctgggccgtgaagcggagccaggagttcgcccagcaggaccccgacaccctgggatggtgggaaaagaacgagatgaacatggtgatgtctctgctcggcatgttctgtcctaccctgttcgacctgtttgccgagctggaggattaccaccctctgatcgccctgaagtggctgctgggtagaatcttcgccctgctgctcggcaacctgtatgtgttcatcctggccctgatggatgagatcaacaataagatcgaagaggaaaagctggtgaaggccaacattaccctgtgggaagccaatatgatcaaggcctataacgcctccttcagcgaaaacagcaccggccctccattcttcgtgcaccccgccgacgtgccaagaggcccttgctgggagacaatggtaggccaggagtttgttagactcacagtgtccgatgtgctgaccacatacgtgaccatcctgatcggcgacttcctgagggcctgcttcgtcagattttgcaactactgctggtgctgggacctggagtacggctaccccagctatacagagttcgacatcagcggcaacgtcctggccctgatctttaaccagggcatgatttggatgggcagcttcttcgcccctagcctgcctggcatcaacatcctgagactgcacacaagcatgtacttccagtgctgggccgtgatgtgttgtaacgtgcccgaggctagagtgttcaaggccagcagaagcaacaacttctacctgggcatgctgcttctgatcctgttcctgtccaccatgcctgtgctgtacatgatcgtgtccctgccacctagcttcgactgcggccctttcagtggaaaaaacagaatgttcgaggtgatcggcgagaccctggagcacgactttccctcttggatggctaagatcctgcggcagctgtctaatcctggcctcgtgatcgccgtgatcctggtcatggtgctggctatctactacctgaacgccaccgctaagggacagaaggccgccaacctggacctgaaaaagaagatgaaaatgcaggccctggaaaacaagatgagaaacaagaagatggccgccgctagagccgctgccgctgctggcagacagtgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - protein, 7 aa, synthetic construct
TLAVPFK

SEQ ID NO: 12 - skipped

SEQ ID NO: 13 - DNA, 8543 bp, synthetic construct
  source 1..8543: mol_type = other DNA
ccaatgatacgcgtcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacatcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatcacatgtcctaggaactatcgatccatcacactggcggccgctcgactagagcggccgccaccgcggtggagctccagcttttgcggaccgaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - protein, 736 aa, synthetic construct
  VARIANT 168: note = K or R
  VARIANT 204: note = A or S
  VARIANT 266: note = A or G
  VARIANT 311: note = R or K
  VARIANT 411: note = E or Q
  VARIANT 460: note = T or E
  VARIANT 493: note = A or T
  VARIANT 562: note = S or N
  VARIANT 576: note = Q or E
  VARIANT 587: note = S or A
  VARIANT 609: note = N or D
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAXKRLNFGQTGDSESVPDPQPLGEPPAAPSGVGSNTMXAGGGAPMADNNEGADGVGNASGNWHCDSTWLGDRVITTSTRTWALPTYNNHLYKQISSQSGXSTNDNTYFGYSTPWGYFDFNRFHCHFSPRDWQRLINNNWGFRPKXLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFXFSYTFEDVPFHSSYAHSQSLDRLMNPLIDQYLYYLSRTQTTSGTAGNRXLQFSQAGPSSMANQAKNWLPGPCYRQQRVSKTXNQNNNSNFAWTGATKYHLNGRDSLVNPGPAMATHKDDEDKFFPMSGVLIFGKQGAGNSNVDLDNVMITXEEEIKTTNPVATEXYGTVATNLQSXNTAPATGTVNSQGALPGMVWQXRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 15 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 16 - DNA, 382 bp, synthetic construct
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 17 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 18 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 19 - skipped

SEQ ID NO: 20 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 21-100 - skipped (80 entries)

SEQ ID NO: 101 - RNA, 13 nt, synthetic construct
  source 1..13: mol_type = other RNA
gccgccaccatgg

SEQ ID NO: 102-103 - skipped (2 entries)

(19) United States
(12) Patent Application Publication
(10) Pub. No.: US 2025/0114480 A1
(43) Pub. Date: Apr. 10, 2025

(54) GENE THERAPY FOR GENETIC HEARING LOSS

(71) Applicant: BridgeBio Gene Therapy Research, Inc., Palo Alto, CA (US)

(72) Inventor: Clayton BEARD, Durham, NC (US)

(21) Appl. No.: 18/833,294

(22) PCT Filed: Jan. 25, 2023
(86) PCT No.: PCT/US2023/061304

§ 371 Date: Jul. 25, 2024

Related U.S. Application Data
(60) Provisional application No. 63/302,913, filed on Jan. 25, 2022.

(51) Int. Cl. A61K 48/00; A61K 9/00; A61K 38/17; A61P 27/02; C12N 15/86

(52) U.S. Cl. CPC A61K 48/005; A61K 9/0046; A61K 38/1709; A61K 48/0075; A61P 27/02; C12N 15/86; C12N 2750/14122; C12N 2750/14143; C12N 2800/22; C12N 2830/48; C12N 2830/50

(57) ABSTRACT

The disclosure provides gene therapy compositions and methods for treating genetic hearing loss (e.g., autosomal recessive non-syndromic hearing loss). In particular, the disclosure provides compositions comprising recombinant adeno-associated viruses (rAAVs) comprising an AAV capsid protein, and an AAV expression cassette encoding transmembrane channel-like 1 (TMC1), and methods of use thereof.

### CROSS REFERENCE TO RELATED APPLICATIONS

The present Application claims the benefit of priority to U.S. Provisional Application No. 63/302,913, filed on Jan. 25, 2022, the contents of which are hereby incorporated by reference in their entirety.

### REFERENCE TO AN ELECTRONIC SEQUENCE LISTING

The contents of sequence the electronic listing (BGTR_004_01WO_SeqList_ST26.xml; Size: 63,410 bytes; and Date of Creation: Jan. 25, 2023) are herein incorporated by reference in their entirety.

### TECHNICAL FIELD

The disclosure relates to adeno-associated virus-mediated gene therapy for genetic hearing loss.

### BACKGROUND

About 50% to 60% of hearing loss in babies is due to genetic causes. Of these, more than 10,000 patients suffer from genetic hearing loss that is associated with the deficiency or dysfunction of transmembrane channel-like 1 (TMC1). Pathophysiology studies show that mutated TMC1 disrupts electrical response to sound-evoked displacement of hair cell stereocilia, and therefore causes moderate to profound sensorineural hearing loss.

Around 60% of patients with TMC1-associated hearing loss experience profound, pre-lingual deafness and are identified through newborn screening. The remaining proportion of patients experience mild-moderate hearing loss in early childhood and progress to severe hearing loss by adolescence. These children often fall in a state of hearing called the “donut hole” where the child is poorly aided but not deaf enough to be indicated for a cochlear implant. These patients are likely to progress to severe or profound deafness. Although patients who have profound hearing loss are indicated for a cochlear implant, these implants often do not fully restore hearing.

Thus, there is an unmet need for compositions and methods, such as gene therapy compositions and methods that can be used to treat genetic hearing loss, such as TMC1-associated hearing loss.

### SUMMARY

The present disclosure provides compositions and methods for use in the treatment of TMC1-associated hearing loss. In an aspect, the present disclosure provides nucleic acid molecules comprising an adeno-associated virus (AAV) expression cassette, wherein the AAV expression cassette comprises, from 5′ to 3′: a 5′ AAV inverted terminal repeat (ITR); a promoter comprising a CB6 promoter; a transgene encoding transmembrane channel-like 1 (TMC1); and a 3′ AAV ITR. The present disclosure also provides plasmids, comprising any one of the nucleic acid molecules disclosed herein, and cells comprising any one of the nucleic acid molecules or plasmids disclosed herein.

The present disclosure also provides recombinant adeno-associated viruses (rAAVs), comprising the AAV expression cassettes disclosed herein, and methods of use thereof in treating a genetic hearing loss in a subject in need thereof, comprising administering to the ear of the subject for an administration period, a therapeutically effective amount of any one of the nucleic acid molecules, plasmids, cells, rAAVs, or compositions disclosed herein.

In some embodiments, the subject suffers from, or is at a risk of developing the genetic hearing loss. In some embodiments, the genetic hearing loss is an autosomal recessive non-syndromic hearing loss (ARNSHL). In some embodiments, the genetic hearing loss is associated with, promoted by, or caused by a mutation in the transmembrane channel-like 1 (TMC1)-encoding gene. In some embodiments, the subject has a hearing threshold in the range of about 25 decibels (dB) to about 80 dB.

In some embodiments, the hearing threshold of the subject during or after the administration period is lower, as compared to before the administration period. In some embodiments, the survival of inner hair cells and/or outer hair cells in the ear of the subject during or after the administration period is improved as compared to the survival of inner hair cells and/or outer hair cells in the ear prior to the administration period.

In some embodiments, the nucleic acid molecule, the plasmid, the cell, the rAAV, or the composition is administered via intracochlear delivery. In some embodiments, the subject is a human subject. In some embodiments, the subject is a neonate or an infant.

### BRIEF DESCRIPTION OF THE DRAWINGS

FIG. 1A-FIG. 1C are graphs showing the auditory brainstem response (ABR; in decibels, dB) obtained at different frequencies (e.g., kilohertz, kHz) in wildtype (WT) mice, TMC1 knockout (KO) mice, or TMC1 KO mice that are 4 weeks (FIG. 1A), 8 weeks (FIG. 1B) or 12 weeks (FIG. 1C) after administration on post-natal day 1 of the indicated dosage (vector genome/cochlea) of a recombinant adeno-associated vector (rAAV), comprising an AAV expression cassette having the nucleic acid sequence of SEQ ID NO: 8. FIG. 1D-FIG. 1E are graphs showing the auditory brainstem response (ABR; in dB) obtained at different frequencies (kHz) in WT mice, TMC1 KO mice, or TMC1 KO mice that are 4 weeks (FIG. 1D) or 8 weeks (FIG. 1E) after administration on post-natal day 1 of the indicated dosage (vector genome/cochlea) of a recombinant adeno-associated vector (rAAV), comprising an AAV expression cassette having the nucleic acid sequence of SEQ ID NO: 9.

FIG. 2A is a graph showing the auditory brainstem response (ABR; in dB) obtained at different frequencies (kHz) in WT mice, TMC1 KO mice, or TMC1 KO mice at the indicated time points after administration on post-natal day 1 of 3.14E10 vector genome/cochlea of a recombinant adeno-associated vector (rAAV), comprising an AAV expression cassette having the nucleic acid sequence of SEQ ID NO: 8. FIG. 2B is a graph showing the auditory brainstem response (ABR; in dB) obtained at different frequencies (kHz) in WT mice, TMC1 KO mice, or TMC1 KO mice at the indicated time points after administration on post-natal day 1 of 3.26E10 vector genome/cochlea of a recombinant adeno-associated vector (rAAV), comprising an AAV expression cassette having the nucleic acid sequence of SEQ ID NO: 10. FIG. 2C is a graph showing the auditory brainstem response (ABR; in dB) obtained at different frequencies (kHz) in WT mice, TMC1 KO mice, or TMC1 KO mice at the indicated time points after administration on post-natal day 1 of 3.14E10 vector genome/cochlea of a recombinant adeno-associated vector (rAAV), comprising an AAV expression cassette having the nucleic acid sequence of SEQ ID NO: 9.

FIG. 3A is a graph showing the auditory brainstem response (ABR; in dB) obtained at different frequencies (kHz) in WT mice, TMC1 KO mice, or TMC1 KO mice at the indicated time points after administration on post-natal day 1 of 7.16E10 vector genome/cochlea of a recombinant adeno-associated vector (rAAV), comprising an AAV expression cassette having the nucleic acid sequence of SEQ ID NO: 8. FIG. 3B is a graph showing the auditory brainstem response (ABR; in dB) obtained at different frequencies (kHz) in WT mice, TMC1 KO mice, or TMC1 KO mice at the indicated time points after administration on post-natal day 1 of 6.3E10 vector genome/cochlea of a recombinant adeno-associated vector (rAAV), comprising an AAV expression cassette having the nucleic acid sequence of SEQ ID NO: 10.

FIG. 4 is a graph that depicts the number of outer hair cells (OHC) per 100 microns in TMC1 KO mice, or TMC1 KO that are 4 weeks after administration on post-natal day 1 of 3E10 vector genome/cochlea of a recombinant adeno-associated vector (rAAV), comprising an AAV expression cassette having the nucleic acid sequence of SEQ ID NO: 8, SEQ ID NO: 9, or SEQ ID NO: 10, as indicated.

FIG. 5A is a graph that depicts the number of inner hair cells (IHC) per 100 microns in TMC1 KO mice, or TMC1 KO that are 4 weeks, 8 weeks, or 12 weeks, or WT mice that are 12 weeks (as indicated) after administration on post-natal day 1 of 3E10 vector genome/cochlea of a recombinant adeno-associated vector (rAAV), comprising an AAV expression cassette having the nucleic acid sequence of SEQ ID NO: 8. FIG. 5B is a graph that depicts the number of outer hair cells (OHC) per 100 microns in TMC1 KO mice, or TMC1 KO that are 4 weeks, 8 weeks, or 12 weeks, or WT mice that are 12 weeks (as indicated) after administration on post-natal day 1 of 3E10 vector genome/cochlea of a recombinant adeno-associated vector (rAAV), comprising an AAV expression cassette having the nucleic acid sequence of SEQ ID NO: 8.

FIG. 6 shows microscopic images showing the morphology of inner hair cells in TMC1 KO 6 months after administration on post-natal day 1 of 7E10 vector genome/cochlea of a recombinant adeno-associated vector (rAAV), comprising an AAV expression cassette having the nucleic acid sequence of SEQ ID NO: 8.

FIG. 7A-FIG. 7B are graphs showing the auditory brainstem response (ABR; in dB) obtained at different frequencies (kHz) in WT mice, N193I mutant mice, or N193I mutant mice at the indicated time points after administration on post-natal day 1 of 3.14E13 vector (FIG. 7A) or 7.16E13 (FIG. 7B) genome/cochlea of a recombinant adeno-associated vector (rAAV), comprising an AAV expression cassette having the nucleic acid sequence of SEQ ID NO: 8. FIG. 7C is a graph showing the auditory brainstem response (ABR; in dB) obtained at different frequencies (kHz) in WT mice, N193I mutant mice, or N193I mutant mice at the indicated time points after administration on post-natal day 1 of 3.78E13 vector genome/cochlea of a recombinant adeno-associated vector (rAAV), comprising an AAV expression cassette having the nucleic acid sequence of SEQ ID NO: 9. FIG. 7D is a graph showing the auditory brainstem response (ABR; in dB) obtained at different frequencies (kHz) in WT mice, N193I mutant mice, or N193I mutant mice at the indicated time points after administration on post-natal day 1 of 3.26E13 vector genome/cochlea of a recombinant adeno-associated vector (rAAV), comprising an AAV expression cassette having the nucleic acid sequence of SEQ ID NO: 10.

FIG. 8A is a graph that depicts the number of inner hair cells (IHC) per 100 microns in WT mice, TMC1 KO mice, N193I mutant mice, or N193I mutant mice that are at the indicated time points after administration on post-natal day 1 of the indicated dosage of a recombinant adeno-associated vector (rAAV), comprising an AAV expression cassette having the nucleic acid sequence of SEQ ID NO: 8. FIG. 8B is a graph that depicts the number of outer hair cells (OHC) per 100 microns in WT mice, TMC1 KO mice, N193I mutant mice, or N193I mutant mice that are at the indicated time points after administration on post-natal day 1 of the indicated dosage of a recombinant adeno-associated vector (rAAV), comprising an AAV expression cassette having the nucleic acid sequence of SEQ ID NO: 8.

FIG. 9A shows microscopic images showing the morphology of hair cells in N193I mutant mice. FIG. 9B shows microscopic images showing the morphology of hair cells in N193I mutant mice that are 6 months after administration on post-natal day 1 of 3E10 vector genome/cochlea of a recombinant adeno-associated vector (rAAV), comprising an AAV expression cassette having the nucleic acid sequence of SEQ ID NO: 8. FIG. 9C shows microscopic images showing the morphology of hair cells in N193I mutant mice that are 12 weeks after administration on post-natal day 1 of 7E10 vector genome/cochlea of a recombinant adeno-associated vector (rAAV), comprising an AAV expression cassette having the nucleic acid sequence of SEQ ID NO: 8. FIG. 9D shows microscopic images showing the morphology of hair cells in N193I mutant mice after administration on post-natal day 1 of 3E10 vector genome/cochlea of a recombinant adeno-associated vector (rAAV), comprising an AAV expression cassette having the nucleic acid sequence of SEQ ID NO: 9.

FIG. 10A-FIG. 10D show zoomed-in versions of the images in FIG. 9A-FIG. 9D, respectively. FIG. 10A shows microscopic images showing the morphology of hair cells in N193I mutant mice. FIG. 10B shows microscopic images showing the morphology of hair cells in N193I mutant mice that are 6 months after administration on post-natal day 1 of 3E10 vector genome/cochlea of a recombinant adeno-associated vector (rAAV), comprising an AAV expression cassette having the nucleic acid sequence of SEQ ID NO: 8. FIG. 10C shows microscopic images showing the morphology of hair cells in N193I mutant mice that are 12 weeks after administration on post-natal day 1 of 7E10 vector genome/cochlea of a recombinant adeno-associated vector (rAAV), comprising an AAV expression cassette having the nucleic acid sequence of SEQ ID NO: 8. FIG. 10D shows microscopic images showing the morphology of hair cells in N193I mutant mice after administration on post-natal day 1 of 3E10 vector genome/cochlea of a recombinant adeno-associated vector (rAAV), comprising an AAV expression cassette having the nucleic acid sequence of SEQ ID NO: 9.

FIG. 11A-FIG. 11C show plasmid maps comprising the AAV expression cassette of SEQ ID NO: 8 (FIG. 11A), SEQ ID NO: 9 (FIG. 11B), and SEQ ID NO: 10 (FIG. 11C).

### DETAILED DESCRIPTION

The present disclosure provides gene therapy compositions and methods for treating genetic hearing loss. In particular, the disclosure provides compositions, comprising recombinant adeno-associated viruses (rAAVs) comprising an AAV capsid protein, and an AAV expression cassette encoding transmembrane channel-like 1 (TMC1), and methods of use thereof.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

The terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%, such as plus or minus 5%.

Also as used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, “treatment,” “treating,” “palliating,” and “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

The term “effective amount” or “therapeutically effective amount” refers to the amount of an agent that is sufficient to achieve an outcome, for example, to effect beneficial or desired results, such as treatment of genetic hearing loss or of a symptom thereof. The therapeutically effective amount may vary depending upon one or more of: the subject and disease condition being treated, the weight and age of the subject, the severity of the disease condition, the manner of administration and the like. A therapeutically effective amount may be an amount sufficient to treat hearing loss and/or to ameliorate, diminish the severity of, eliminate, and/or delay the onset of one or more symptoms of genetic hearing loss.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, a subject is an infant or neonate. In some embodiments, a subject is up to 24 months old. In some embodiments, a subject is less than 6 years of age. In some embodiments, a subject is between about 6 and about 12 years of age. In some embodiments, a subject is between about 12 and about 18 years of age. In some embodiments, a subject is less than 18 years of age. In some embodiments, a subject is at least 18 years of age. In some embodiments, the subject is a pregnant woman or a woman intending to become pregnant.

An “adeno-associated virus (AAV) expression cassette” is a nucleic acid that gets packaged into a recombinant AAV vector, and comprises a sequence encoding one or more transgenes, such as one or more transgenes flanked by a 5′ inverted terminal repeat (ITR) and a 3′ITR.

As used herein, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors include adeno-associated virus vectors (AAVs).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, AAV9-php.b, and any other AAV now known or later discovered.

As used herein “sequence identity” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539 For the purposes of calculating identity to a sequence, extensions such as tags are not included.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein.

As used herein, the hearing threshold is the sound level below which a person's ear is unable to detect any sound. The hearing threshold of a control subject exhibiting “normal hearing” is in the range of about 0 dB to about 15 dB.

As used herein, “genetic hearing loss” refers to an impairment or reduction of hearing in a subject and a higher hearing threshold in the subject, as compared to a control subject exhibiting normal hearing. In some embodiments, the genetic hearing loss is associated with, promoted by, or caused by a dysfunction of the inner ear, the outer ear, or a combination thereof. In some embodiments, genetic hearing loss is progressive (e.g., worsens over time). In some embodiments, genetic hearing loss is non-progressive (e.g., does not worsen over time). In some embodiments, genetic hearing loss is familial. In some embodiments, genetic hearing loss is sporadic or de novo. In some embodiments, genetic hearing loss is syndromic (e.g., associated with one or more other conditions). In some embodiments, genetic hearing loss is non-syndromic (e.g., not-associated with other conditions). In some embodiments, genetic hearing loss is acquired. In some embodiments, genetic hearing loss is congenital (e.g., present at birth). In some embodiments, the genetic hearing loss is associated with one or more mutations (e.g., recessive mutations) of TMC1.

In some embodiments, a subject with a hearing threshold of about 25 dB to about 40 dB is said to be experiencing “mild hearing loss”. In some embodiments, a subject with a hearing threshold of about 40 dB to about 55 dB is said to be experiencing “moderate hearing loss”. In some embodiments, a subject with a hearing threshold of about 55 dB to about 70 dB is said to be experiencing “moderate to severe hearing loss”. In some embodiments, a subject with a hearing threshold of about 70 dB to about 90 dB is said to be experiencing “severe hearing loss”. In some embodiments, a subject with a hearing threshold of about 90 dB to about 120 dB is said to be experiencing “profound hearing loss”.

In the present disclosure, dosages may be presented using scientific notation, which can be converted into a decimal number by a person of ordinary skill in the art. For instance, a dosage written in the scientific notation of 310E refers to a decimal number of 3λ10¹⁰.

### AAV Expression Cassettes Encoding Transmembrane Channel-Like 1 (TMC1)

The present disclosure provides gene therapy compositions and methods for treating genetic hearing loss (e.g., autosomal recessive non-syndromic hearing loss (ARNSHL)). For example, the present disclosure provides nucleic acid molecules comprising adeno-associated virus (AAV) expression cassettes. In some embodiments, the AAV expression cassette of a nucleic acid molecule comprises, from 5′ to 3′: a 5′ AAV inverted terminal repeat (ITR); a promoter; a transgene (e.g., a transgene encoding transmembrane channel-like 1 (TMC1)); and a 3′ AAV ITR.

In some embodiments, the AAV expression cassette comprises cis-acting 5′ and 3′ inverted terminal repeat sequences, as described further in B. J. Carter, in “Handbook of Parvoviruses”, ed., P. Tijsser, CRC Press, pp. 155 168 (1990), which is incorporated herein by reference in its entirety for all purposes. The AAV ITR sequences may be obtained from any known or presently unknown AAV, including presently identified mammalian AAV types disclosed herein.

In some embodiments, the AAV expression cassette comprises a 5′ ITR and/or a 3′ ITR derived from AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LK03, AAV7m8, AAV Anc80, or AAV9-php.b. In some embodiments, the AAV expression cassette comprises a 5′ ITR derived from AAV2, a 3′ ITR derived from AAV2, or a combination thereof.

In some embodiments, the 5′ AAV ITR sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 6. In some embodiments, the 5′ AAV ITR sequence comprises, or consists of, the sequence of SEQ ID NO: 6.

In some embodiments, the 3′ AAV ITR sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 7. In some embodiments, the 3′ AAV ITR sequence comprises, or consists of, the sequence of SEQ ID NO: 7.

In some embodiments, the AAV expression cassette comprises expression control elements which are operably linked to the transgene. Expression control elements include appropriate transcription initiation, termination, and enhancer sequences; efficient RNA processing signals such as splicing and polyadenylation (polyA) signals; sequences that stabilize cytoplasmic mRNA; sequences that enhance translation efficiency; sequences that enhance protein stability; and, in some cases, sequences that enhance secretion of the encoded product.

In some embodiments, the AAV expression cassette comprises an intron. In some embodiments, the intron is located between a promoter/enhancer sequence and a transgene. In some embodiments, the intron is derived from SV-40, and is referred to as the SV-40 T intron sequence. In some embodiments, the AAV expression cassette comprises an internal ribosome entry site (IRES). In some embodiments, the AAV expression cassette comprises a nucleic acid encoding a 2A self-cleaving peptide. Illustrative 2A self-cleaving peptides include P2A, E2A, F2A, and T2A. In some embodiments, the AAV expression cassette comprises an element described in Sambrook et al., Molecular Cloning: A Laboratory Manual, Cold Spring Harbor Press, Cold Spring Harbor, N.Y., and references cited therein, at, for example, pages 3.18, 3.26, 16.17, and 16.27 and Ausubel et al., Current Protocols in Molecular Biology, John Wiley & Sons, New York, 1989, each of which is incorporated herein by reference in its entirety for all purposes.

In some embodiments, the AAV expression cassette comprises a woodchuck hepatitis virus post-transcriptional element (WPRE). (See, e.g., Wang and Verma, Proc. Natl. Acad. Sci., USA, 96:3906-3910 (1999)). In some embodiments, the AAV expression cassette comprises a hepatitis B virus posttranscriptional regulatory element (HBVPRE) and/or a RNA transport element (RTE). In some embodiments, the WPRE or HBVPRE sequence is any of the WPRE or HBVPRE sequences disclosed in U.S. Pat. Nos. 6,136,597 and 6,287,814. In some embodiments, the WPRE comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 5. In some embodiments, the WPRE comprises the nucleic acid sequence of SEQ ID NO: 5.

In some embodiments, the AAV expression cassette comprises one or more 5′-non-transcribed and/or 5′-non-translated sequences involved with the initiation of transcription and translation, respectively, such as a TATA box, capping sequence, CAAT sequence, enhancer element, or the like. In some embodiments, the AAV expression cassette comprises an enhancer sequence and/or upstream activator sequence. In some embodiments, the AAV expression cassette comprises one or more 5′ leader and/or signal sequences.

In some embodiments, the AAV expression cassette comprises one or more promoters. In some embodiments, the AAV expression cassette comprises a chicken β-actin promoter. In some embodiments, the AAV expression cassette comprises a CB6 promoter. In some embodiments, the CB6 promoter comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 2. In some embodiments, the CB6 promoter comprises, or consists of, the nucleic acid sequence of SEQ ID NO: 2.

In some embodiments, the AAV expression cassette comprises a CMV promoter. In some embodiments, the CMV promoter comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 18. In some embodiments, the CMV promoter comprises, or consists of, the nucleic acid sequence of SEQ ID NO: 18.

In some embodiments, the AAV expression cassette comprises a promoter selected from an Espin promoter, a protocadherin 15 (PCDH15) promoter, a PTPRQ promoter, a My06 promoter, a KCNQ4 promoter, a myosin 7a (Myo7a) promoter, a synapsin promoter, a GFAP promoter, a CMV promoter, a CAG promoter, a CBH promoter, a CBA promoter, a U6 promoter, and a TMHS (LHFPL5) promoter.

In some embodiments, the AAV expression cassette comprises a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the CMV-IE enhancer, the SV40 promoter, the dihydrofolate reductase promoter, the β-actin promoter, the phosphoglycerol kinase (PGK) promoter, and the EF1a promoter.

In some embodiments, the AAV expression cassette comprises an inducible promoter. Non-limiting examples of inducible promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system, and the rapamycin-inducible system. Other types of inducible promoters include those that are regulated by a specific physiological state, e.g., temperature, acute phase, a particular differentiation state of the cell, or a specific cell cycle phase.

In some embodiments, the AAV expression cassette comprises the native promoter, or fragment thereof, or the native expression control element, operably linked to the transgene encoding TMC1. In some embodiments, the AAV expression cassette comprises one or more regulatory sequences that impart tissue-specific gene expression capabilities (e.g., tissue-specific regulatory sequences). In some cases, a tissue-specific regulatory sequence binds one or more tissue-specific transcription factors that induce transcription in a tissue-specific manner. Examples of tissue-specific regulatory sequences include, but are not limited to, the following tissue specific promoters: neuronal promoters such as the neuron-specific enolase (NSE) promoter, the neurofilament light chain gene promoter, and the neuron-specific vgf gene promoter.

In some embodiments, the AAV expression cassette comprises a CMV-IE enhancer. In some embodiments, the enhancer is a CMV-IE enhancer. In some embodiments, the CMV-IE enhancer comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 16 or SEQ ID NO: 17. In some embodiments, the CMV-IE enhancer comprises, or consists of, the nucleic acid sequence of SEQ ID NO: 16 or SEQ ID NO: 17.

In some embodiments, the AAV expression cassette comprises a consensus sequence, such as a Kozak sequence (for example, a DNA sequence transcribed to an RNA Kozak sequence). As used herein, a “Kozak sequence” refers to a DNA element encoding an “RNA Kozak sequence” which regulates translational initiation.” In some embodiments, the AAV expression cassette comprises a Kozak sequence. In some embodiments, the Kozak sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 4. In some embodiments, the Kozak sequence comprises, or consists of, the nucleic acid sequence of SEQ ID NO: 4.

In some embodiments, the AAV expression cassette comprises a Kozak sequence upstream of the transgene. In some embodiments, the Kozak sequence (e.g., RNA Kozak sequence) comprises or consists of ACCAUGG (SEQ ID NO: 100), GCCGCCACCAUGG (SEQ ID NO: 101), CCACCAUG (SEQ ID NO: 102), or CCACCAUGG (SEQ ID NO: 103).

In some embodiments, the AAV expression cassette comprises one or more binding sites for one or more miRNAs. Furthermore, the TMC1 transgene may be designed such that multiple miRNAs regulate mRNA by recognizing the same or multiple sites. The presence of multiple miRNA binding sites may result in the cooperative action of multiple RNA-induced silencing complexes (RISCs) and provide highly efficient inhibition of expression. The target site sequence may comprise a total of at least 5, 10, or more nucleotides, such as between 5-100, or between 10-60 nucleotides. The target site sequence may comprise at least 5 nucleotides of the sequence of a target gene binding site. In some embodiments, the AAV expression cassette comprises an miR-1 binding site, an miR-133a binding site, an miR-122 binding site, or any combination thereof. Further details on the miRNAs are provided in Geisler and Fechner, World J Exp Med 2016 May 20; 6 (2): 37-54, which is incorporated herein by reference in its entirety for all purposes.

In some embodiments, the AAV expression cassette comprises a polyadenylation (poly A) sequence. As used herein, the “polyA sequence” refers to a DNA sequence that when transcribed regulates the addition of a polyA tail to the mRNA transcript. PolyA signals may be derived from many suitable species, including, without limitation SV-40, human, and bovine. In some embodiments, the polyA sequence is a β-globin polyA sequence, such as a mammalian β-globin polyA sequence. In some embodiments, the poly A sequence is a human polyA sequence or a bovine β-globin polyA sequence. In some embodiments, the AAV expression cassette comprises a rabbit β-globin polyA sequence. In some embodiments, the β-globin poly A sequence comprises the nucleic acid sequence of SEQ ID NO: 3.

In some embodiments, the transgene comprises a sequence encoding the TMC1 protein. In some embodiments, the transgene comprises a codon-optimized sequence encoding the TMC1 protein. In some embodiments, the transgene comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 1. In some embodiments, the transgene comprises a codon-optimized sequence comprising or consisting of the nucleic acid sequence of SEQ ID NO: 1.

In some embodiments, the AAV expression cassette comprises, from 5′ to 3′: (i) a 5′ ITR (e.g. an AAV2-based ITR), (ii) a CB6 promoter, (iii) a Kozak sequence, (iv) a codon optimized transgene encoding human TMC1 protein, (v) a woodchuck hepatitis virus posttranscriptional regulatory element (WPRE), (vi) a beta globin polyadenylation sequence, and (vii) a 3′ ITR (e.g. an AAV2-based ITR).

In some embodiments, the AAV expression cassette comprises, from 5′ to 3′: (i) a 5′ ITR comprising the nucleic acid sequence of SEQ ID NO: 6, (ii) a CB6 promoter comprising the nucleic acid sequence of SEQ ID NO: 2, (iii) a Kozak sequence comprising the nucleic acid sequence of SEQ ID NO: 4, (iv) a codon optimized transgene encoding human TMC1 protein comprising the nucleic acid sequence of SEQ ID NO: 1, (v) a woodchuck hepatitis virus posttranscriptional regulatory element (WPRE) comprising the nucleic acid sequence of SEQ ID NO: 5, (vi) a beta globin polyadenylation sequence comprising the nucleic acid sequence of SEQ ID NO: 3, and (vii) a 3′ ITR comprising the nucleic acid sequence of SEQ ID NO: 7.

In some embodiments, the AAV expression cassette comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 8. In some embodiments, the AAV expression cassette comprises or consists of the nucleic acid sequence of SEQ ID NO: 8.

In some embodiments, the AAV expression cassette comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 9. In some embodiments, the AAV expression cassette comprises or consists of the nucleic acid sequence of SEQ ID NO: 9.

In some embodiments, the AAV expression cassette comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 10. In some embodiments, the AAV expression cassette comprises or consists of the nucleic acid sequence of SEQ ID NO: 10.

Recombinant Adeno-Associated Virus (rAAV) for Treating Genetic Hearing Loss

The present disclosure also provides plasmids, comprising any one of the nucleic acid molecules disclosed herein, and cells comprising any one of the nucleic acid molecules or plasmids disclosed herein.

The present disclosure further provides methods of producing a recombinant adeno-associated virus (rAAV). In some embodiments, a method of producing an rAAV comprises contacting an AAV producer cell with any one of the nucleic acid molecules or plasmids disclosed herein. Accordingly, the present disclosure also provides recombinant adeno-associated viruses (rAAVs) produced by the methods of producing rAAVs disclosed herein. In some embodiments, the rAAV comprises an AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, or AAV9-php.b capsid protein.

In some embodiments, the rAAV comprises an AAV9 capsid protein, an AAV Anc80 capsid protein, and/or an AAV9-php.b capsid protein. In some embodiments, the rAAV comprises an AAV9 capsid protein. In some embodiments, the rAAV comprises an AAV9-php.b capsid protein.

In some embodiments, the AAV9-php.b capsid protein is encoded by a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 13. In some embodiments, the AAV9-php.b capsid protein is encoded by the nucleic acid sequence of SEQ ID NO: 13.

In some embodiments, the AAV9-Anc80 capsid protein has an amino acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 14 or SEQ ID NO: 15. In some embodiments, the AAV9-Anc80 capsid protein has the amino acid sequence of SEQ ID NO: 14 or SEQ ID NO: 15. Further details on the AAV9-php.b and AAV Anc80 capsids are provided in WO2019173367A1, WO2018145111A1, and WO2017100791A1, the contents of which are herein incorporated by reference in their entirety for all purposes.

The present disclosure also provides recombinant adeno-associated viruses (rAAVs). In some embodiments, an rAAV comprises an AAV9-php.b capsid protein and any one of the nucleic acid molecules disclosed herein. In some embodiments, an rAAV comprises an AAV9-php.b capsid protein and a nucleic acid molecule, wherein the nucleic acid molecule comprises an AAV expression cassette, wherein the AAV expression cassette comprises, from 5′ to 3′: (i) a 5′ ITR (e.g. an AAV2-based ITR), (ii) a CB6 promoter, (iii) a Kozak sequence, (iv) a codon optimized transgene encoding human TMC1 protein, (v) a woodchuck hepatitis virus posttranscriptional regulatory element (WPRE), (vi) a beta globin polyadenylation sequence, and (vii) a 3′ ITR (e.g. an AAV2-based ITR).

In some embodiments, an rAAV comprises an AAV9-php.b capsid protein and a nucleic acid molecule, wherein the nucleic acid molecule comprises an AAV expression cassette, wherein the AAV expression cassette comprises, from 5′ to 3′: (i) a 5′ ITR comprising the nucleic acid sequence of SEQ ID NO: 6, (ii) a CB6 promoter comprising the nucleic acid sequence of SEQ ID NO: 2, (iii) a Kozak sequence comprising the nucleic acid sequence of SEQ ID NO: 4, (iv) a codon optimized transgene encoding human TMC1 protein comprising the nucleic acid sequence of SEQ ID NO: 1, (v) a woodchuck hepatitis virus posttranscriptional regulatory element (WPRE) comprising the nucleic acid sequence of SEQ ID NO: 5, (vi) a beta globin polyadenylation sequence comprising the nucleic acid sequence of SEQ ID NO: 3, and (vii) a 3′ ITR comprising the nucleic acid sequence of SEQ ID NO: 7.

In some embodiments, an rAAV comprises an AAV9-php.b capsid protein; and a nucleic acid molecule, wherein the nucleic acid molecule comprises an AAV expression cassette, wherein the AAV expression cassette comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 8. In some embodiments, an rAAV comprises an AAV9-php.b capsid protein; and a nucleic acid molecule, wherein the nucleic acid molecule comprises an AAV expression cassette, wherein the AAV expression cassette comprises a codon-optimized nucleic acid sequence of SEQ ID NO: 8.

In some embodiments, an rAAV is a self-complementary AAV. In some embodiments, an rAAV is a single-stranded AAV.

In some embodiments, preparation of rAAV particles involves culturing a host cell that contains a nucleic acid sequence encoding an AAV capsid protein or fragment thereof; a functional rep gene; a recombinant AAV vector composed of AAV inverted terminal repeats (ITRs) and the AAV expression cassette encoding TMC1; and sufficient helper functions to permit packaging of the recombinant AAV vector into the AAV capsid proteins. In some embodiments, the components to be cultured in the host cell to package a rAAV vector in an AAV capsid are provided to the host cell in trans. In some embodiments, any one or more of the required components (e.g., recombinant AAV vector, rep sequences, cap sequences, and/or helper functions) are provided by a stable host cell that has been engineered to contain one or more of the required components. In some embodiments, a stable host cell will contain the required component(s) under the control of an inducible promoter or a constitutive promoter. In some embodiments, a selected stable host cell contains selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. The recombinant AAV vector, rep sequences, cap sequences, and helper functions required for producing the rAAVs disclosed herein may be delivered to the packaging host cell using any appropriate genetic element (for example, a vector). Further details on methods of preparing rAAV particles are provided in Sambrook et al., Molecular Cloning: A Laboratory Manual, Cold Spring Harbor Press, Cold Spring Harbor, N.Y.; K. Fisher et al, J. Virol., 70:520-532 (1993) and U.S. Pat. No. 5,478,745, the contents of each of which are herein incorporated in its entirety for all purposes.

In some embodiments, recombinant AAVs are produced using the triple transfection method, as described in U.S. Pat. No. 6,001,650, the contents of which are herein incorporated in its entirety for all purposes. In some embodiments, the recombinant AAVs are produced by transfecting a host cell with a recombinant AAV vector (comprising the AAV expression cassette encoding TMC1) to be packaged into AAV particles, an AAV helper function vector, and an accessory function vector. An AAV helper function vector encodes the “AAV helper function” sequences (i.e., rep and cap), which function in trans for productive AAV replication and encapsidation. Non-limiting examples of AAV helper function vectors include pHLP19 and pRep6cap6 vector, described in U.S. Pat. Nos. 6,001,650 and 6,156,303, respectively, the contents of each of which are herein incorporated in its entirety for all purposes. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, recombinant AAVs are produced using baculovirus vectors. Baculovirus vectors are used to produce recombinant AAVs in insect cells (e.g., Spodoptera frugiperda (Sf9) cells).

### Pharmaceutical Compositions

The present disclosure further provides pharmaceutical compositions, comprising: (a) any one of the nucleic acid molecules disclosed herein, any one of the plasmids disclosed herein, any one of the cells disclosed herein, or any one the rAAVs disclosed herein; and (b) a pharmaceutically acceptable carrier.

In some embodiments, the compositions disclosed herein comprise at least one pharmaceutically acceptable carrier, excipient, and/or vehicle, for example, solvents, buffers, solutions, dispersion media, coatings, antibacterial agents, antifungal agents, isotonic agents, and absorption delaying agents. In some embodiments, the pharmaceutically acceptable carrier, excipient, and/or vehicle comprises saline, buffered saline, dextrose, water, glycerol, sterile isotonic aqueous buffer, or a combination thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like), or a suitable mixture thereof. In some embodiments, the compositions disclosed herein further comprise emulsifying or wetting agents, or pH buffering agents. Such species may be present in small amounts (e.g., less than 10% by weight of the composition, such as less than 5% by weight of the composition, 2% by weight of the composition, 1% by weight of the composition, or less).

In some embodiments, the compositions disclosed herein further comprise one or more other pharmaceutical ingredients, such as one or more preservatives or chemical stabilizers. Examples of preservatives and chemical stabilizers include, but are not limited to, chlorobutanol, potassium sorbate, sorbic acid, sulfur dioxide, propyl gallate, parabens, ethyl vanillin, glycerin, phenol, parachlorophenol, and albumin. In some embodiments, the compositions disclosed herein further comprise antibacterial agents and/or antifungal agents, such as parabens, chlorobutanol, phenol, sorbic acid, and thimerosal; isotonic agents, such as sugars and sodium chloride; and/or agents delaying absorption, such as aluminum monostearate and gelatin.

In some embodiments, the compositions disclosed herein are formulated to reduce aggregation of AAV particles in the composition, particularly where high rAAV concentrations are present (e.g., ˜10¹³ GC/mL or more). Methods for reducing aggregation of rAAVs include addition of surfactants, pH adjustment, and salt concentration adjustment, as further described in Wright, et al., Molecular Therapy (2005) 12, 171-178, the contents of which are incorporated herein by reference in its entirety for all purposes.

In some embodiments, the pharmaceutical compositions are in a form of an injectable solution or dispersion, such as an aqueous solution or dispersion. In some embodiments, the pharmaceutical composition is a sterile powder for the extemporaneous preparation of sterile injectable solutions or dispersions. Dispersions may be prepared in water, glycerol, liquid polyethylene glycols, oils, or any combination thereof. Delivery vehicles such as liposomes, nanocapsules, microparticles, microspheres, lipid particles, vesicles, and the like, may be used for the introduction of the pharmaceutical compositions disclosed herein.

### Methods of Treating Genetic Hearing Loss

The present disclosure also provides methods of expressing transmembrane channel-like 1 (TMC1) in a cell, comprising: contacting any one of the nucleic acid molecules disclosed herein, any one of the plasmids disclosed herein, any one of the rAAVs disclosed herein, or any one of the compositions disclosed herein with the cell, thereby expressing TMC1 in the cell. In some embodiments, the cell is an ear cell. In some embodiments, the cell is an inner hair cell (IHC), or an outer hair cell (OHC). In some embodiments, the cell is a vestibular hair cell, spiral ganglion, or vestibular ganglion.

In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the rAAV, or the composition to the subject.

The present disclosure further provides methods of treating genetic hearing loss or a symptom thereof in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby treating genetic hearing loss or a symptom thereof in the subject. In a related aspect, the present disclosure provides methods of ameliorating, diminishing the severity of, eliminating, and/or delaying the onset of one or more symptoms of genetic hearing loss in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of genetic hearing loss in the subject.

In some embodiments, the subject suffers from, or is at a risk of developing the genetic hearing loss. In some embodiments, the genetic hearing loss is associated with one or more mutations (e.g., recessive mutations) in TMC1. In some embodiments, the genetic hearing loss is an autosomal recessive non-syndromic hearing loss (ARNSHL). In some embodiments, the subject has autosomal dominant non-syndromic sensorineural hearing loss. In some embodiments, the subject has a mutant allele of TMC1 called DFNA36. In some embodiments, the subject has autosomal recessive non-syndromic neurosensory deafness. In some embodiments, the subject has a mutant allele of TMC1 called DFNB7. In some embodiments, the genetic hearing loss is associated with, promoted by, or caused by a mutation in the transmembrane channel-like 1 (TMC1)-encoding gene. In some embodiments, the mutation in the TMC1-encoding gene is present at the DFNB7/11 locus on chromosome 9q31-21.

In some embodiments, the method comprises diminishing the severity of; delaying the onset or progression of; and/or eliminating a symptom of the genetic hearing loss. In some embodiments, the symptom of the genetic hearing loss comprises: a reduced ability to hear, or an inability to hear. In some embodiments, the genetic hearing loss is: (a) a progressive genetic hearing loss, (b) a pre-lingual genetic hearing loss, (c) a congenital genetic hearing loss, or (d) any combination thereof. In some embodiments, the subject has a hearing threshold in the range of about 25 dB to about 80 dB. In some embodiments, the subject has a hearing threshold of about 40 dB. In some embodiments, the subject has a hearing threshold of about 55 dB. In some embodiments, the subject has a hearing threshold of about 70 dB.

In some embodiments, the method comprises decreasing the hearing threshold of the subject during or after the administration period, as compared to prior to the administration period. In some embodiments, the hearing threshold of the subject during or after the administration period is lower, as compared to before the administration period. In some embodiments, the hearing threshold of the subject during or after the administration period is at least about 2% (for example, about 5%, about 10%, about 15%, about 20%, about 30%, about 40%, about 50%, about 60%, about 70%, about 80%, about 90%, or 100%, including values and subranges that lie therebetween) lower, as compared to before the administration period. In some embodiments, the hearing threshold of the subject during or after the administration period is at least about 5% lower, as compared to before the administration period.

In some embodiments, the hearing threshold during or after the administration period of a subject, who is administered an rAAV, comprising: an AAV9-php.b capsid protein; and a nucleic acid molecule, comprising an AAV expression cassette, wherein the AAV expression cassette comprises, from 5′ to 3′: a 5′ AAV ITR, a CB6 promoter, a Kozak sequence, a transgene, encoding a codon-optimized human transmembrane channel-like 1 (co-hTMC1), a woodchuck hepatitis virus posttranscriptional regulatory element (WPRE), a beta globin polyadenylation sequence, and a 3′ AAV ITR, is lower, as compared to that of a control subject, wherein the control subject is administered an rAAV, comprising: an AAV9-php.b capsid protein; and a nucleic acid molecule, comprising an AAV expression cassette, wherein the AAV expression cassette lacks a woodchuck hepatitis virus posttranscriptional regulatory element (WPRE) and comprises, from 5′ to 3′: a 5′ AAV ITR, a CB6 promoter, a Kozak sequence, a transgene, encoding a codon-optimized human transmembrane channel-like 1 (co-hTMC1), a beta globin polyadenylation sequence, and a 3′ AAV ITR.

In some embodiments, the hearing threshold during or after the administration period of a subject, who is administered an rAAV, comprising: an AAV9-php.b capsid protein; and a nucleic acid molecule, comprising an AAV expression cassette, wherein the AAV expression cassette comprises, from 5′ to 3′: a 5′ AAV ITR, a CB6 promoter, a Kozak sequence, a transgene, encoding a codon-optimized human transmembrane channel-like 1 (co-hTMC1), a woodchuck hepatitis virus posttranscriptional regulatory element (WPRE), a beta globin polyadenylation sequence, and a 3′ AAV ITR, is lower for a longer period of time, as compared to a control subject, wherein the control subject is administered an rAAV, comprising: an AAV9-php.b capsid protein; and a nucleic acid molecule, comprising an AAV expression cassette, wherein the AAV expression cassette lacks a woodchuck hepatitis virus posttranscriptional regulatory element (WPRE) and comprises, from 5′ to 3′: a 5′ AAV ITR, a CB6 promoter, a Kozak sequence, a transgene, encoding a codon-optimized human transmembrane channel-like 1 (co-hTMC1), a beta globin polyadenylation sequence, and a 3′ AAV ITR.

In some embodiments, the hearing threshold during or after the administration period of a subject, who is administered an rAAV, comprising: an AAV9-php.b capsid protein; and a nucleic acid molecule, comprising an AAV expression cassette, wherein the AAV expression cassette comprises, from 5′ to 3′: a 5′ AAV ITR, a CB6 promoter, a Kozak sequence, a transgene, encoding a codon-optimized human transmembrane channel-like 1 (co-hTMC1), a woodchuck hepatitis virus posttranscriptional regulatory element (WPRE), a beta globin polyadenylation sequence, and a 3′ AAV ITR, is lower, as compared to that of a control subject, wherein the control subject is administered an rAAV, comprising: an AAV9-php.b capsid protein; and a nucleic acid molecule, comprising an AAV expression cassette, wherein the AAV expression cassette lacks a CB6 promoter and comprises, from 5′ to 3′: a 5′ AAV ITR, a CMV promoter, a Kozak sequence, a transgene, encoding a codon-optimized human transmembrane channel-like 1 (co-hTMC1), a beta globin polyadenylation sequence, a woodchuck hepatitis virus posttranscriptional regulatory element (WPRE) and a 3′ AAV ITR.

In some embodiments, the hearing threshold during or after the administration period of the subject, who is administered an rAAV, comprising: an AAV9-php.b capsid protein; and a nucleic acid molecule, comprising an AAV expression cassette, wherein the AAV expression cassette comprises, from 5′ to 3′: a 5′ AAV ITR, a CB6 promoter, a Kozak sequence, a transgene, encoding a codon-optimized human transmembrane channel-like 1 (co-hTMC1), a woodchuck hepatitis virus posttranscriptional regulatory element (WPRE), a beta globin polyadenylation sequence, and a 3′ AAV ITR, is lower for a longer period of time, as compared to that of a control subject, wherein the control subject is administered an rAAV, comprising: an AAV9-php.b capsid protein; and a nucleic acid molecule, comprising an AAV expression cassette, wherein the AAV expression cassette lacks a CB6 promoter and comprises, from 5′ to 3′: a 5′ AAV ITR, a CMV promoter, a Kozak sequence, a transgene, encoding a codon-optimized human transmembrane channel-like 1 (co-hTMC1), a beta globin polyadenylation sequence, a woodchuck hepatitis virus posttranscriptional regulatory element (WPRE) and a 3′ AAV ITR.

In some embodiments, the survival of inner hair cells and/or outer hair cells in the ear of the subject during or after the administration period is improved as compared to the survival of inner hair cells and/or outer hair cells in the ear prior to the administration period. In some embodiments, the number of inner hair cells and/or outer hair cells in the ear of the subject during or after the administration period is higher, as compared to the number of inner hair cells and/or outer hair cells in the ear before the administration period.

In some embodiments of any of the preceding aspects, the administration is intravenous or intravascular. In some embodiments, the administration is by direct delivery to the ear, or a component thereof. In some embodiments of any of the preceding aspects, the administration is by intracochlear injection, or any other mode of administration into the ear, or a mode of administration that is suitable for expression of the transgene in the ear. In some embodiments of any of the preceding aspects, the administration is by intrathecal administration.

In some embodiments of any of the preceding aspects, the administration is by injection into the central nervous system. Other modes of administration that may be used include transmucosal, intranasal, oral, inhalation (e.g., via an aerosol), buccal (e.g., sublingual), intrathecal, intraocular, transdermal, parenteral (e.g., intravenous, subcutaneous, intradermal, intramuscular, intradermal, intrapleural, intracerebral, and intraarticular), topical (e.g., to skin and/or mucosal surfaces, including airway surfaces, and transdermal administration), intralymphatic, and the like, as well as direct tissue injection (e.g., to the ear).

In some embodiments of any of the preceding aspects, the method comprises administering a therapeutically effective amount of an rAAV, wherein the therapeutically effective amount is in a range of about 10⁵ genome copies to 10²⁰ genome copies per cochlea administered, for example, about 10⁶ genome copies/cochlea, about 10⁷ genome copies/cochlea, about 10⁸ genome copies/cochlea, about 10⁹ genome copies/cochlea, about 10¹⁰ genome copies/cochlea, about 10¹¹ genome copies/cochlea, about 10¹² genome copies/cochlea, about 10¹³ genome copies/cochlea, about 10¹⁴ genome copies/cochlea, about 10¹⁵ genome copies/cochlea, about 10¹⁶ genome copies/cochlea, about 10¹⁷ genome copies/cochlea, about 10¹⁸ genome copies/cochlea, or about 10¹⁹ genome copies/cochlea, including all values and subranges that lie therebetween. In some embodiments, the method comprises administering a therapeutically effective amount of rAAV, wherein the therapeutically effective amount is in a range of 10¹⁰ genome copies to 10¹⁴ genome copies per kilogram.

In some embodiments of any of the preceding aspects, the method comprises administering a therapeutically effective amount of an rAAV, wherein the therapeutically effective amount is in a range of about 10⁵ genome copies to 10²⁰ genome copies per kilogram (kg), for example, about 10⁶ genome copies/kg, about 10⁷ genome copies/kg, about 10⁸ genome copies/kg, about 10⁹ genome copies/kg, about 10¹⁰ genome copies/kg, about 10¹¹ genome copies/kg, about 10¹² genome copies/kg, about 10¹³ genome copies/kg, about 10¹⁴ genome copies/kg, about 10¹⁵ genome copies/kg, about 10¹⁶ genome copies/kg, about 10¹⁷ genome copies/kg, about 10¹⁸ genome copies/kg, or about 10¹⁹ genome copies/kg, including all values and subranges that lie therebetween. In some embodiments, the method comprises administering a therapeutically effective amount of rAAV, wherein the therapeutically effective amount is in a range of 10¹⁰ genome copies to 10¹⁴ genome copies per kilogram.

In some embodiments of any of the preceding aspects, the therapeutically effective amount is in the range of about 10⁵ to 10²⁰ genome copies per subject, for example, about 10⁶ genome copies per subject, about 10⁷ genome copies per subject, about 10⁸ genome copies per subject, about 10⁹ genome copies per subject, about 10¹⁰ genome copies per subject, about 10¹¹ genome copies per subject, about 10¹² genome copies per subject, about 10¹³ genome copies per subject, about 10¹⁴ genome copies per subject, about 10¹⁵ genome copies per subject, about 10¹⁶ genome copies per subject, about 10¹⁷ genome copies per subject, about 10¹⁸ genome copies per subject, or about 10¹⁹ genome copies per subject, including all values and subranges that lie therebetween. In some embodiments, the therapeutically effective amount is in the range of about 10⁹ to 10¹⁶ genome copies per subject.

In some embodiments of any of the preceding aspects, the therapeutically effective amount is administered in a volume of about 1 microliter (μl) to about 100 milliliter (mL) of solution, for example, about 10 μl, about 50 μl, about 100 μl, about 500 μl, about 1 mL, about 20 mL, about 30 mL, about 40 mL, about 50 mL, about 60 mL, about 70 mL, about 80 mL, about 90 mL, or about 100 mL, including all values and subranges that lie therebetween. The volume used may depend on the dose of the rAAV, and the route of administration. For example, for intrathecal administration a volume in the range of about 1 μl to about 10 μl, or about 10 μl to about 100 μl may be used. For intravenous administration a volume in range of about 10 μl to about 100 μl, or about 100 μl to 1 mL, or about 1 mL to about 10 mL, or more may be used.

In some embodiments of any of the preceding aspects, more than one administration (e.g., two, three, four or more administrations) may be employed to achieve the desired level of gene expression over a period of various intervals, e.g., daily, weekly, monthly, yearly, etc.

In some embodiments, the subject is a human subject. In some embodiments, the subject is a neonate or an infant. In some embodiments, a subject is up to 24 months old. In some embodiments, a subject is less than 6 years of age. In some embodiments, a subject is between about 6 and about 12 years of age. In some embodiments, a subject is between about 12 and about 18 years of age. In some embodiments, the subject is less than 18 years of age. In some embodiments, the subject is at least 18 years of age.

The present disclosure also provides kits comprising one or more agents (e.g., any one of the nucleic acid molecules disclosed herein, any one of the plasmids disclosed herein, any one of the rAAVs disclosed herein, or any one of the compositions disclosed herein). In some embodiments, the kits are pharmaceutical or diagnostic or research kits to be used in therapeutic, diagnostic or research applications. A kit may include one or more containers housing the agents disclosed herein and instructions for use. In certain embodiments, agents in a kit are in a pharmaceutical formulation and dosage suitable for a particular application and for a method of administration of the agents. In some embodiments, the container is a syringe, vial, tube, topical application devices, IV needle tubing and bag, and other containers.

Provided below are the nucleic acid and protein sequences disclosed in this application:

Sequences

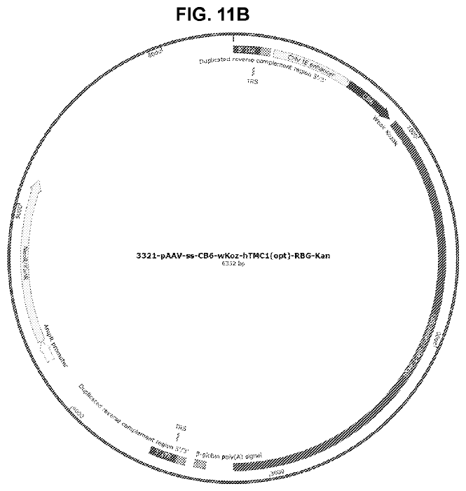

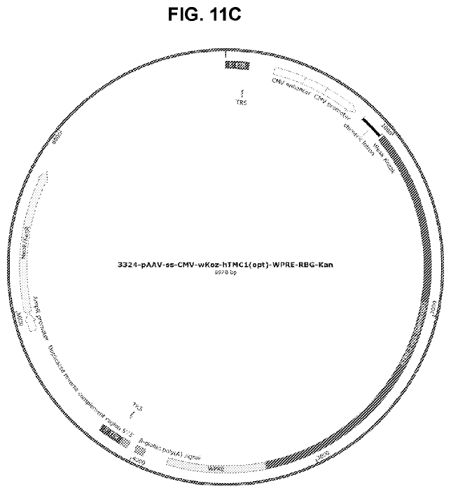

SEQ ID NO: 8-Expression cassette of plasmid depicted in FIG. 11A
CTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCC
AACTCCATCACTAGGGGTTCCTTGTAGTTAATGATTAACCCGCCATGCTACTTAT
CTACCAGGGTAATGGGGATCCTCTAGAACTATAGCTAGTCGACATTGATTATTGA
CTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGC
AGTACATCTACGTATTAGTCATCGCTATTACcatgtcgaggccacgttctgcttcactctccccatctcccc
cccctccccacccccaattttgtatttatttattttttaattattttgtgcagcgatgggggcggggggggggggcgcgcgccaggcggg
gcggggcggggcgaggggcggggcggggcgaggcggagaggtgcggcggcagccaatcagagcggcgcgctccgaaagttt
ccttttatggcgaggcggcggcggcggcggccctataaaaagcgaagcgcgcggcgggcgggagcaagctctagcctcgagaatt
cccgcggATGAGCCCTAAGAAGGTGCAGATCAAGGTGGAAGAAAAGGAGGACGAG
ACAGAAGAGAGCTCTTCTGAAGAGGAAGAGGAAGTGGAAGATAAGCTGCCACG
GAGAGAAAGCCTGCGCCCCAAGAGAAAGAGAACCAGAGATGTGATCAACGAGG
ACGATCCTGAACCTGAGCCTGAGGACGAGGAAACAAGAAAAGCACGAGAGAAG
GAACGGCGGAGAAGGCTGAAGCGGGGCGCTGAGGAAGAAGAAATCGACGAGGA
GGAGCTGGAAAGACTTAAGGCCGAGCTGGATGAGAAGCGCCAAATCATCGCCAC
AGTGAAATGCAAGCCCTGGAAGATGGAAAAGAAAATCGAGGTGCTGAAGGAGG
CCAAAAAGTTCGTCTCCGAGAATGAGGGCGCCCTCGGTAAGGGCAAGGGCAAGC
GGTGGTTCGCCTTCAAGATGATGATGGCTAAGAAGTGGGCCAAGTTCCTGCGGG
ACTTCGAGAATTTCAAGGCCGCTTGTGTGCCTTGGGAAAACAAGATTAAGGCAA
TCGAGAGCCAATTTGGAAGCAGCGTGGCCTCCTACTTTCTGTTTCTGAGATGGAT
GTACGGAGTGAACATGGTGCTGTTCATCCTGACATTTAGCCTGATCATGCTGCCT
GAGTACCTATGGGGCCTGCCCTACGGCAGCCTGCCTCGGAAGACCGTGCCTCGG
GCCGAAGAGGCCAGCGCCGCCAACTTCGGCGTGCTGTACGACTTCAACGGCCTT
GCTCAGTACAGCGTGCTGTTTTACGGCTACTACGACAACAAACGGACCATCGGCT
GGATGAACTTCAGACTGCCTCTGAGCTACTTCCTGGTGGGCATCATGTGCATCGG
ATACTCTTTCCTGGTTGTGCTCAAAGCCATGACCAAGAACATCGGCGACGACGGC
GGAGGCGATGATAACACCTTCAATTTTTCTTGGAAAGTGTTCACCAGCTGGGACT
ACCTGATTGGAAACCCTGAGACAGCCGATAATAAGTTCAATAGCATCACCATGA
ACTTCAAGGAAGCCATCACCGAGGAGAAAGCCGCCCAAGTGGAAGAAAACGTTC
ATCTGATCAGATTCCTGAGATTCCTGGCCAACTTTTTCGTGTTCCTGACCCTGGGC
GGAAGCGGCTACCTGATCTTCTGGGCCGTGAAGCGGAGCCAGGAGTTCGCCCAG
CAGGACCCCGACACCCTGGGATGGTGGGAAAAGAACGAGATGAACATGGTGAT
GTCTCTGCTCGGCATGTTCTGTCCTACCCTGTTCGACCTGTTTGCCGAGCTGGAGG
ATTACCACCCTCTGATCGCCCTGAAGTGGCTGCTGGGTAGAATCTTCGCCCTGCT
GCTCGGCAACCTGTATGTGTTCATCCTGGCCCTGATGGATGAGATCAACAATAAG
ATCGAAGAGGAAAAGCTGGTGAAGGCCAACATTACCCTGTGGGAAGCCAATATG
ATCAAGGCCTATAACGCCTCCTTCAGCGAAAACAGCACCGGCCCTCCATTCTTCG
TGCACCCCGCCGACGTGCCAAGAGGCCCTTGCTGGGAGACAATGGTAGGCCAGG
AGTTTGTTAGACTCACAGTGTCCGATGTGCTGACCACATACGTGACCATCCTGAT
CGGCGACTTCCTGAGGGCCTGCTTCGTCAGATTTTGCAACTACTGCTGGTGCTGG
GACCTGGAGTACGGCTACCCCAGCTATACAGAGTTCGACATCAGCGGCAACGTC
CTGGCCCTGATCTTTAACCAGGGCATGATTTGGATGGGCAGCTTCTTCGCCCCTA
GCCTGCCTGGCATCAACATCCTGAGACTGCACACAAGCATGTACTTCCAGTGCTG
GGCCGTGATGTGTTGTAACGTGCCCGAGGCTAGAGTGTTCAAGGCCAGCAGAAG
CAACAACTTCTACCTGGGCATGCTGCTTCTGATCCTGTTCCTGTCCACCATGCCTG
TGCTGTACATGATCGTGTCCCTGCCACCTAGCTTCGACTGCGGCCCTTTCAGTGG
AAAAAACAGAATGTTCGAGGTGATCGGCGAGACCCTGGAGCACGACTTTCCCTC
TTGGATGGCTAAGATCCTGCGGCAGCTGTCTAATCCTGGCCTCGTGATCGCCGTG
ATCCTGGTCATGGTGCTGGCTATCTACTACCTGAACGCCACCGCTAAGGGACAGA
AGGCCGCCAACCTGGACCTGAAAAAGAAGATGAAAATGCAGGCCCTGGAAAAC
AAGATGAGAAACAAGAAGATGGCCGCCGCTAGAGCCGCTGCCGCTGCTGGCAGA
CAGTGAGgatccaatcaacctctggattacaaaatttgtgaaagattgactggtattcttaactatgttgctccttttacgctatgtgga
tacgctgctttaatgcctttgtatcatgctattgcttcccgtatggctttcattttctcctccttgtataaatcctggttgctgtctctttatg
aggagttgtggcccgttgtcaggcaacgtggcgtggtgtgcactgtgtttgctgacgcaacccccactggttggggcattgccaccacctgtc
agctcctttccgggactttcgctttccccctccctattgccacggeggaactcatcgccgcctgccttgcccgctgctggacaggggctc
ggctgttgggcactgacaattccgtggtgttgtcggggaaatcatcgtcctttccttggctgctcgcctgtgttgccacctggattctgcgc
gggacgtccttctgctacgtcccttcggccctcaatccagcggaccttccttcccgeggcctgctgceggctctgcggcctcttccgcgt
cttcgACGCGTGGTACCTCTAGAGTCGACCCGGGCGGCCTCGAGGACGGGGTGAAC
TACGCCTGAGGATCCGATCTTTTTCCCTCTGCCAAAAATTATGGGGACATCATGA
AGCCCCTTGAGCATCTGACTTCTGGCTAATAAAGGAAATTTATTTTCATTGCAAT
AGTGTGTTGGAATTTTTTGTGTCTCTCACTCGGAAGCAATTCGTTGATCTGAATTT
CGACCACCCATAATACCCATTACCCTGGTAGATAAGTAGCATGGCGGGTTAATCA
TTAACTACAAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCG
GGCGGCCTCAGTGAGCGAGCGAGCGCGCAG
Elements of SEQ ID NO: 8:
5′ ITR-SEQ ID NO: 6
CTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCC
AACTCCATCACTAGGGGTTCCT
CMV IE enhancer-SEQ ID NO: 16
CTAGTCGACATTGATTATTGACTAGTTATTAATAGTAATCAATTACGGGGTCATT
AGTTCATAGCCCATATATGGAGTTCCGCGTTACATAACTTACGGTAAATGGCCCG
CCTGGCTGACCGCCCAACGACCCCCGCCCATTGACGTCAATAATGACGTATGTTC
CCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCT
ATTGACGTCAATGACGGTAAATGGCCCGCCTGGCATTATGCCCAGTACATGACCT
TATGGGACTTTCCTACTTGGCAGTACATCTACGTATTAGTCATCGCTATTAC
CB6-SEQ ID NO: 2
ccacgttctgcttcactctccccatctcccccccctccccacccccaattttgtatttatttattttttaattattttgtgcagcgatgggggc
ggggggggggggcgcgcgccaggcggggcggggcggggcgaggggcggggcggggcgaggcggagaggtgcggcggcagc
caatcagagcggcgcgctccgaaagtttccttttatggcgaggcggcggcggcggcggccctataaaaagcgaagcgcgcgggg
g
Weak Kozak-SEQ ID NO: 4
ccgcgg
hTMC1-opt-SEQ ID NO: 1
ATGAGCCCTAAGAAGGTGCAGATCAAGGTGGAAGAAAAGGAGGACGAGACAGA
AGAGAGCTCTTCTGAAGAGGAAGAGGAAGTGGAAGATAAGCTGCCACGGAGAG
AAAGCCTGCGCCCCAAGAGAAAGAGAACCAGAGATGTGATCAACGAGGACGAT
CCTGAACCTGAGCCTGAGGACGAGGAAACAAGAAAAGCACGAGAGAAGGAACG
GCGGAGAAGGCTGAAGCGGGGCGCTGAGGAAGAAGAAATCGACGAGGAGGAGC
TGGAAAGACTTAAGGCCGAGCTGGATGAGAAGCGCCAAATCATCGCCACAGTGA
AATGCAAGCCCTGGAAGATGGAAAAGAAAATCGAGGTGCTGAAGGAGGCCAAA
AAGTTCGTCTCCGAGAATGAGGGCGCCCTCGGTAAGGGCAAGGGCAAGCGGTGG
TTCGCCTTCAAGATGATGATGGCTAAGAAGTGGGCCAAGTTCCTGCGGGACTTCG
AGAATTTCAAGGCCGCTTGTGTGCCTTGGGAAAACAAGATTAAGGCAATCGAGA
GCCAATTTGGAAGCAGCGTGGCCTCCTACTTTCTGTTTCTGAGATGGATGTACGG
AGTGAACATGGTGCTGTTCATCCTGACATTTAGCCTGATCATGCTGCCTGAGTAC
CTATGGGGCCTGCCCTACGGCAGCCTGCCTCGGAAGACCGTGCCTCGGGCCGAA
GAGGCCAGCGCCGCCAACTTCGGCGTGCTGTACGACTTCAACGGCCTTGCTCAGT
ACAGCGTGCTGTTTTACGGCTACTACGACAACAAACGGACCATCGGCTGGATGA
ACTTCAGACTGCCTCTGAGCTACTTCCTGGTGGGCATCATGTGCATCGGATACTC
TTTCCTGGTTGTGCTCAAAGCCATGACCAAGAACATCGGCGACGACGGCGGAGG
CGATGATAACACCTTCAATTTTTCTTGGAAAGTGTTCACCAGCTGGGACTACCTG
ATTGGAAACCCTGAGACAGCCGATAATAAGTTCAATAGCATCACCATGAACTTC
AAGGAAGCCATCACCGAGGAGAAAGCCGCCCAAGTGGAAGAAAACGTTCATCT
GATCAGATTCCTGAGATTCCTGGCCAACTTTTTCGTGTTCCTGACCCTGGGCGGA
AGCGGCTACCTGATCTTCTGGGCCGTGAAGCGGAGCCAGGAGTTCGCCCAGCAG
GACCCCGACACCCTGGGATGGTGGGAAAAGAACGAGATGAACATGGTGATGTCT
CTGCTCGGCATGTTCTGTCCTACCCTGTTCGACCTGTTTGCCGAGCTGGAGGATTA
CCACCCTCTGATCGCCCTGAAGTGGCTGCTGGGTAGAATCTTCGCCCTGCTGCTC
GGCAACCTGTATGTGTTCATCCTGGCCCTGATGGATGAGATCAACAATAAGATCG
AAGAGGAAAAGCTGGTGAAGGCCAACATTACCCTGTGGGAAGCCAATATGATCA
AGGCCTATAACGCCTCCTTCAGCGAAAACAGCACCGGCCCTCCATTCTTCGTGCA
CCCCGCCGACGTGCCAAGAGGCCCTTGCTGGGAGACAATGGTAGGCCAGGAGTT
TGTTAGACTCACAGTGTCCGATGTGCTGACCACATACGTGACCATCCTGATCGGC
GACTTCCTGAGGGCCTGCTTCGTCAGATTTTGCAACTACTGCTGGTGCTGGGACC
TGGAGTACGGCTACCCCAGCTATACAGAGTTCGACATCAGCGGCAACGTCCTGG
CCCTGATCTTTAACCAGGGCATGATTTGGATGGGCAGCTTCTTCGCCCCTAGCCT
GCCTGGCATCAACATCCTGAGACTGCACACAAGCATGTACTTCCAGTGCTGGGCC
GTGATGTGTTGTAACGTGCCCGAGGCTAGAGTGTTCAAGGCCAGCAGAAGCAAC
AACTTCTACCTGGGCATGCTGCTTCTGATCCTGTTCCTGTCCACCATGCCTGTGCT
GTACATGATCGTGTCCCTGCCACCTAGCTTCGACTGCGGCCCTTTCAGTGGAAAA
AACAGAATGTTCGAGGTGATCGGCGAGACCCTGGAGCACGACTTTCCCTCTTGG
ATGGCTAAGATCCTGCGGCAGCTGTCTAATCCTGGCCTCGTGATCGCCGTGATCC
TGGTCATGGTGCTGGCTATCTACTACCTGAACGCCACCGCTAAGGGACAGAAGG
CCGCCAACCTGGACCTGAAAAAGAAGATGAAAATGCAGGCCCTGGAAAACAAG
ATGAGAAACAAGAAGATGGCCGCCGCTAGAGCCGCTGCCGCTGCTGGCAGACAG
TGA
WPRE-SEQ ID NO: 5
aatcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggcccgt
tgtcaggcaacgtggcgtggtgtgcactgtgtttgctgacgcaacccccactggttggggcattgccaccacctgtcagctcctttccgggact
ttcgctttccccctccctattgccacggcggaactcatcgccgcctgccttgcccgctgctggacaggggctcggctgttgggcactga
caattccgtggtgttgtcggggaaatcatcgtcctttccttggctgctcgcctgtgttgccacctggattctgcgcgggacgtccttctgct
acgtcccttcggccctcaatccagcggaccttccttcccgcggcctgctgccggctctgcggcctcttccgcgtcttcg
Beta-globin poly(A) signal-SEQ ID NO: 3
AATAAAGGAAATTTATTTTCATTGCAATAGTGTGTTGGAATTTTTTGTGTCTCTCA
3′ ITR-SEQ ID NO: 7
AGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTC
AGTGAGCGAGCGAGCGCGCAG
SEQ ID NO: 9-Expression cassette of plasmid depicted in FIG. 11B
CTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCC
AACTCCATCACTAGGGGTTCCTTGTAGTTAATGATTAACCCGCCATGCTACTTAT
CTACCAGGGTAATGGGGATCCTCTAGAACTATAGCTAGTCGACATTGATTATTGA
CTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGC
AGTACATCTACGTATTAGTCATCGCTATTACcatgtcgaggccacgttctgcttcactctccccatctcccc
cccctccccacccccaattttgtatttatttattttttaattattttgtgcagcgatgggggcggggggggggggcgcgcgccaggcggg
gcggggcggggcgaggggcggggcggggcgaggcggagaggtgcggcggcagccaatcagagcggcgcgctccgaaagttt
ccttttatggcgaggcggcggcggcggcggccctataaaaagcgaagcgcgcggcgggcgggagcaagctctagcctcgagaatt
cccgcggATGAGCCCTAAGAAGGTGCAGATCAAGGTGGAAGAAAAGGAGGACGAG
ACAGAAGAGAGCTCTTCTGAAGAGGAAGAGGAAGTGGAAGATAAGCTGCCACG
GAGAGAAAGCCTGCGCCCCAAGAGAAAGAGAACCAGAGATGTGATCAACGAGG
ACGATCCTGAACCTGAGCCTGAGGACGAGGAAACAAGAAAAGCACGAGAGAAG
GAACGGCGGAGAAGGCTGAAGCGGGGCGCTGAGGAAGAAGAAATCGACGAGGA
GGAGCTGGAAAGACTTAAGGCCGAGCTGGATGAGAAGCGCCAAATCATCGCCAC
AGTGAAATGCAAGCCCTGGAAGATGGAAAAGAAAATCGAGGTGCTGAAGGAGG
CCAAAAAGTTCGTCTCCGAGAATGAGGGCGCCCTCGGTAAGGGCAAGGGCAAGC
GGTGGTTCGCCTTCAAGATGATGATGGCTAAGAAGTGGGCCAAGTTCCTGCGGG
ACTTCGAGAATTTCAAGGCCGCTTGTGTGCCTTGGGAAAACAAGATTAAGGCAA
TCGAGAGCCAATTTGGAAGCAGCGTGGCCTCCTACTTTCTGTTTCTGAGATGGAT
GTACGGAGTGAACATGGTGCTGTTCATCCTGACATTTAGCCTGATCATGCTGCCT
GAGTACCTATGGGGCCTGCCCTACGGCAGCCTGCCTCGGAAGACCGTGCCTCGG
GCCGAAGAGGCCAGCGCCGCCAACTTCGGCGTGCTGTACGACTTCAACGGCCTT
GCTCAGTACAGCGTGCTGTTTTACGGCTACTACGACAACAAACGGACCATCGGCT
GGATGAACTTCAGACTGCCTCTGAGCTACTTCCTGGTGGGCATCATGTGCATCGG
ATACTCTTTCCTGGTTGTGCTCAAAGCCATGACCAAGAACATCGGCGACGACGGC
GGAGGCGATGATAACACCTTCAATTTTTCTTGGAAAGTGTTCACCAGCTGGGACT
ACCTGATTGGAAACCCTGAGACAGCCGATAATAAGTTCAATAGCATCACCATGA
ACTTCAAGGAAGCCATCACCGAGGAGAAAGCCGCCCAAGTGGAAGAAAACGTTC
ATCTGATCAGATTCCTGAGATTCCTGGCCAACTTTTTCGTGTTCCTGACCCTGGGC
GGAAGCGGCTACCTGATCTTCTGGGCCGTGAAGCGGAGCCAGGAGTTCGCCCAG
CAGGACCCCGACACCCTGGGATGGTGGGAAAAGAACGAGATGAACATGGTGAT
GTCTCTGCTCGGCATGTTCTGTCCTACCCTGTTCGACCTGTTTGCCGAGCTGGAGG
ATTACCACCCTCTGATCGCCCTGAAGTGGCTGCTGGGTAGAATCTTCGCCCTGCT
GCTCGGCAACCTGTATGTGTTCATCCTGGCCCTGATGGATGAGATCAACAATAAG
ATCGAAGAGGAAAAGCTGGTGAAGGCCAACATTACCCTGTGGGAAGCCAATATG
ATCAAGGCCTATAACGCCTCCTTCAGCGAAAACAGCACCGGCCCTCCATTCTTCG
TGCACCCCGCCGACGTGCCAAGAGGCCCTTGCTGGGAGACAATGGTAGGCCAGG
AGTTTGTTAGACTCACAGTGTCCGATGTGCTGACCACATACGTGACCATCCTGAT
CGGCGACTTCCTGAGGGCCTGCTTCGTCAGATTTTGCAACTACTGCTGGTGCTGG
GACCTGGAGTACGGCTACCCCAGCTATACAGAGTTCGACATCAGCGGCAACGTC
CTGGCCCTGATCTTTAACCAGGGCATGATTTGGATGGGCAGCTTCTTCGCCCCTA
GCCTGCCTGGCATCAACATCCTGAGACTGCACACAAGCATGTACTTCCAGTGCTG
GGCCGTGATGTGTTGTAACGTGCCCGAGGCTAGAGTGTTCAAGGCCAGCAGAAG
CAACAACTTCTACCTGGGCATGCTGCTTCTGATCCTGTTCCTGTCCACCATGCCTG
TGCTGTACATGATCGTGTCCCTGCCACCTAGCTTCGACTGCGGCCCTTTCAGTGG
AAAAAACAGAATGTTCGAGGTGATCGGCGAGACCCTGGAGCACGACTTTCCCTC
TTGGATGGCTAAGATCCTGCGGCAGCTGTCTAATCCTGGCCTCGTGATCGCCGTG
ATCCTGGTCATGGTGCTGGCTATCTACTACCTGAACGCCACCGCTAAGGGACAGA
AGGCCGCCAACCTGGACCTGAAAAAGAAGATGAAAATGCAGGCCCTGGAAAAC
AAGATGAGAAACAAGAAGATGGCCGCCGCTAGAGCCGCTGCCGCTGCTGGCAGA
CAGTGAACGCGTGGTACCTCTAGAGTCGACCCGGGCGGCCTCGAGGACGGGGTG
AACTACGCCTGAGGATCCGATCTTTTTCCCTCTGCCAAAAATTATGGGGACATCA
TGAAGCCCCTTGAGCATCTGACTTCTGGCTAATAAAGGAAATTTATTTTCATTGC
AATAGTGTGTTGGAATTTTTTGTGTCTCTCACTCGGAAGCAATTCGTTGATCTGAA
TTTCGACCACCCATAATACCCATTACCCTGGTAGATAAGTAGCATGGGGGTTAA
TCATTAACTACAAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGC
CCGGGCGGCCTCAGTGAGCGAGCGAGCGCGCAG
Elements of SEQ ID NO: 9:
5′ ITR-SEQ ID NO: 6
CTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCC
AACTCCATCACTAGGGGTTCCT
CMV IE enhancer-SEQ ID NO: 16
CTAGTCGACATTGATTATTGACTAGTTATTAATAGTAATCAATTACGGGGTCATT
AGTTCATAGCCCATATATGGAGTTCCGCGTTACATAACTTACGGTAAATGGCCCG
CCTGGCTGACCGCCCAACGACCCCCGCCCATTGACGTCAATAATGACGTATGTTC
CCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCT
ATTGACGTCAATGACGGTAAATGGCCCGCCTGGCATTATGCCCAGTACATGACCT
TATGGGACTTTCCTACTTGGCAGTACATCTACGTATTAGTCATCGCTATTAC
CB6-SEQ ID NO: 2
ccacgttctgcttcactctccccatctcccccccctccccacccccaattttgtatttatttattttttaattattttgtgcagcgatgggggc
ggggggggggggcgcgcgccaggcggggcggggcggggcgaggggcggggcggggcgaggcggagaggtgcggcggcagc
caatcagagcggcgcgctccgaaagtttccttttatggcgaggcggcggcggcggcggccctataaaaagcgaagcgcgcggcgg
g
Weak Kozak-SEQ ID NO: 4
ccgcgg
hTMC1-opt-SEQ ID NO: 1
ATGAGCCCTAAGAAGGTGCAGATCAAGGTGGAAGAAAAGGAGGACGAGACAGA
AGAGAGCTCTTCTGAAGAGGAAGAGGAAGTGGAAGATAAGCTGCCACGGAGAG
AAAGCCTGCGCCCCAAGAGAAAGAGAACCAGAGATGTGATCAACGAGGACGAT
CCTGAACCTGAGCCTGAGGACGAGGAAACAAGAAAAGCACGAGAGAAGGAACG
GCGGAGAAGGCTGAAGCGGGGCGCTGAGGAAGAAGAAATCGACGAGGAGGAGC
TGGAAAGACTTAAGGCCGAGCTGGATGAGAAGCGCCAAATCATCGCCACAGTGA
AATGCAAGCCCTGGAAGATGGAAAAGAAAATCGAGGTGCTGAAGGAGGCCAAA
AAGTTCGTCTCCGAGAATGAGGGCGCCCTCGGTAAGGGCAAGGGCAAGCGGTGG
TTCGCCTTCAAGATGATGATGGCTAAGAAGTGGGCCAAGTTCCTGCGGGACTTCG
AGAATTTCAAGGCCGCTTGTGTGCCTTGGGAAAACAAGATTAAGGCAATCGAGA
GCCAATTTGGAAGCAGCGTGGCCTCCTACTTTCTGTTTCTGAGATGGATGTACGG
AGTGAACATGGTGCTGTTCATCCTGACATTTAGCCTGATCATGCTGCCTGAGTAC
CTATGGGGCCTGCCCTACGGCAGCCTGCCTCGGAAGACCGTGCCTCGGGCCGAA
GAGGCCAGCGCCGCCAACTTCGGCGTGCTGTACGACTTCAACGGCCTTGCTCAGT
ACAGCGTGCTGTTTTACGGCTACTACGACAACAAACGGACCATCGGCTGGATGA
ACTTCAGACTGCCTCTGAGCTACTTCCTGGTGGGCATCATGTGCATCGGATACTC
TTTCCTGGTTGTGCTCAAAGCCATGACCAAGAACATCGGCGACGACGGCGGAGG
CGATGATAACACCTTCAATTTTTCTTGGAAAGTGTTCACCAGCTGGGACTACCTG
ATTGGAAACCCTGAGACAGCCGATAATAAGTTCAATAGCATCACCATGAACTTC
AAGGAAGCCATCACCGAGGAGAAAGCCGCCCAAGTGGAAGAAAACGTTCATCT
GATCAGATTCCTGAGATTCCTGGCCAACTTTTTCGTGTTCCTGACCCTGGGCGGA
AGCGGCTACCTGATCTTCTGGGCCGTGAAGCGGAGCCAGGAGTTCGCCCAGCAG
GACCCCGACACCCTGGGATGGTGGGAAAAGAACGAGATGAACATGGTGATGTCT
CTGCTCGGCATGTTCTGTCCTACCCTGTTCGACCTGTTTGCCGAGCTGGAGGATTA
CCACCCTCTGATCGCCCTGAAGTGGCTGCTGGGTAGAATCTTCGCCCTGCTGCTC
GGCAACCTGTATGTGTTCATCCTGGCCCTGATGGATGAGATCAACAATAAGATCG
AAGAGGAAAAGCTGGTGAAGGCCAACATTACCCTGTGGGAAGCCAATATGATCA
AGGCCTATAACGCCTCCTTCAGCGAAAACAGCACCGGCCCTCCATTCTTCGTGCA
CCCCGCCGACGTGCCAAGAGGCCCTTGCTGGGAGACAATGGTAGGCCAGGAGTT
TGTTAGACTCACAGTGTCCGATGTGCTGACCACATACGTGACCATCCTGATCGGC
GACTTCCTGAGGGCCTGCTTCGTCAGATTTTGCAACTACTGCTGGTGCTGGGACC
TGGAGTACGGCTACCCCAGCTATACAGAGTTCGACATCAGCGGCAACGTCCTGG
CCCTGATCTTTAACCAGGGCATGATTTGGATGGGCAGCTTCTTCGCCCCTAGCCT
GCCTGGCATCAACATCCTGAGACTGCACACAAGCATGTACTTCCAGTGCTGGGCC
GTGATGTGTTGTAACGTGCCCGAGGCTAGAGTGTTCAAGGCCAGCAGAAGCAAC
AACTTCTACCTGGGCATGCTGCTTCTGATCCTGTTCCTGTCCACCATGCCTGTGCT
GTACATGATCGTGTCCCTGCCACCTAGCTTCGACTGCGGCCCTTTCAGTGGAAAA
AACAGAATGTTCGAGGTGATCGGCGAGACCCTGGAGCACGACTTTCCCTCTTGG
ATGGCTAAGATCCTGCGGCAGCTGTCTAATCCTGGCCTCGTGATCGCCGTGATCC
TGGTCATGGTGCTGGCTATCTACTACCTGAACGCCACCGCTAAGGGACAGAAGG
CCGCCAACCTGGACCTGAAAAAGAAGATGAAAATGCAGGCCCTGGAAAACAAG
ATGAGAAACAAGAAGATGGCCGCCGCTAGAGCCGCTGCCGCTGCTGGCAGACAG
TGA
Beta-globin poly(A) signal-SEQ ID NO: 3
AATAAAGGAAATTTATTTTCATTGCAATAGTGTGTTGGAATTTTTTGTGTCTCTCA
3′ ITR-SEQ ID NO: 7
AGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTC
AGTGAGCGAGCGAGCGCGCAG
SEQ ID NO: 10-Expression cassette of plasmid depicted in FIG. 11C
CTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCC
AACTCCATCACTAGGGGTTCCTtgtagttaatgattaacccgccatgctacttatctacgtagccatgctctaggaag
atcggaattcgcccttaagctagctagttattaatagtaatcaattacggggtcattagttcatagcccatatatggagttccgcgttacata
acttacggtaaatggcccgcctggctgaccgcccaacgacccccgcccattgacgtcaataatgacgtatgttcccatagtaacgcca
atagggactttccattgacgtcaatgggtggagtatttacggtaaactgcccacttggcagtacatcaagtgtatCatatgccaagtacg
ccccctattgacgtcaatgacggtaaatggcccgcctggcattatgcccagtacatgaccttatgggactttcctacttggcagtacatct
acgtattagtcatcgctattaccatggtgatgcggttttggcagtacatcaatgggcgtggatagcggtttgactcacggggatttccaag
tctccaccccattgacgtcaatgggagtttgttttggcaccaaaatcaacgggactttccaaaatgtcgtaacaactccgccccattgacg
caaatgggcggtaggcgtgtacggtgggaggtctatataagcagagctggtttagtgaaccgtcagatcctgcagaagttggtcgtga
ggcactgggcaggtaagtatcaaggttacaagacaggtttaaggagaccaatagaaactgggcttgtcgagacagagaagactcttg
cgtttctgataggcacctattggtcttactgacatccactttgcctttctctccacaggtgtccaggcggccgcggATGAGCCCT
AAGAAGGTGCAGATCAAGGTGGAAGAAAAGGAGGACGAGACAGAAGAGAGCTC
TTCTGAAGAGGAAGAGGAAGTGGAAGATAAGCTGCCACGGAGAGAAAGCCTGC
GCCCCAAGAGAAAGAGAACCAGAGATGTGATCAACGAGGACGATCCTGAACCT
GAGCCTGAGGACGAGGAAACAAGAAAAGCACGAGAGAAGGAACGGCGGAGAA
GGCTGAAGCGGGGCGCTGAGGAAGAAGAAATCGACGAGGAGGAGCTGGAAAGA
CTTAAGGCCGAGCTGGATGAGAAGCGCCAAATCATCGCCACAGTGAAATGCAAG
CCCTGGAAGATGGAAAAGAAAATCGAGGTGCTGAAGGAGGCCAAAAAGTTCGT
CTCCGAGAATGAGGGCGCCCTCGGTAAGGGCAAGGGCAAGCGGTGGTTCGCCTT
CAAGATGATGATGGCTAAGAAGTGGGCCAAGTTCCTGCGGGACTTCGAGAATTT
CAAGGCCGCTTGTGTGCCTTGGGAAAACAAGATTAAGGCAATCGAGAGCCAATT
TGGAAGCAGCGTGGCCTCCTACTTTCTGTTTCTGAGATGGATGTACGGAGTGAAC
ATGGTGCTGTTCATCCTGACATTTAGCCTGATCATGCTGCCTGAGTACCTATGGG
GCCTGCCCTACGGCAGCCTGCCTCGGAAGACCGTGCCTCGGGCCGAAGAGGCCA
GCGCCGCCAACTTCGGCGTGCTGTACGACTTCAACGGCCTTGCTCAGTACAGCGT
GCTGTTTTACGGCTACTACGACAACAAACGGACCATCGGCTGGATGAACTTCAG
ACTGCCTCTGAGCTACTTCCTGGTGGGCATCATGTGCATCGGATACTCTTTCCTGG
TTGTGCTCAAAGCCATGACCAAGAACATCGGCGACGACGGCGGAGGCGATGATA
ACACCTTCAATTTTTCTTGGAAAGTGTTCACCAGCTGGGACTACCTGATTGGAAA
CCCTGAGACAGCCGATAATAAGTTCAATAGCATCACCATGAACTTCAAGGAAGC
CATCACCGAGGAGAAAGCCGCCCAAGTGGAAGAAAACGTTCATCTGATCAGATT
CCTGAGATTCCTGGCCAACTTTTTCGTGTTCCTGACCCTGGGCGGAAGCGGCTAC
CTGATCTTCTGGGCCGTGAAGCGGAGCCAGGAGTTCGCCCAGCAGGACCCCGAC
ACCCTGGGATGGTGGGAAAAGAACGAGATGAACATGGTGATGTCTCTGCTCGGC
ATGTTCTGTCCTACCCTGTTCGACCTGTTTGCCGAGCTGGAGGATTACCACCCTCT
GATCGCCCTGAAGTGGCTGCTGGGTAGAATCTTCGCCCTGCTGCTCGGCAACCTG
TATGTGTTCATCCTGGCCCTGATGGATGAGATCAACAATAAGATCGAAGAGGAA
AAGCTGGTGAAGGCCAACATTACCCTGTGGGAAGCCAATATGATCAAGGCCTAT
AACGCCTCCTTCAGCGAAAACAGCACCGGCCCTCCATTCTTCGTGCACCCCGCCG
ACGTGCCAAGAGGCCCTTGCTGGGAGACAATGGTAGGCCAGGAGTTTGTTAGAC
TCACAGTGTCCGATGTGCTGACCACATACGTGACCATCCTGATCGGCGACTTCCT
GAGGGCCTGCTTCGTCAGATTTTGCAACTACTGCTGGTGCTGGGACCTGGAGTAC
GGCTACCCCAGCTATACAGAGTTCGACATCAGCGGCAACGTCCTGGCCCTGATCT
TTAACCAGGGCATGATTTGGATGGGCAGCTTCTTCGCCCCTAGCCTGCCTGGCAT
CAACATCCTGAGACTGCACACAAGCATGTACTTCCAGTGCTGGGCCGTGATGTGT
TGTAACGTGCCCGAGGCTAGAGTGTTCAAGGCCAGCAGAAGCAACAACTTCTAC
CTGGGCATGCTGCTTCTGATCCTGTTCCTGTCCACCATGCCTGTGCTGTACATGAT
CGTGTCCCTGCCACCTAGCTTCGACTGCGGCCCTTTCAGTGGAAAAAACAGAATG
TTCGAGGTGATCGGCGAGACCCTGGAGCACGACTTTCCCTCTTGGATGGCTAAGA
TCCTGCGGCAGCTGTCTAATCCTGGCCTCGTGATCGCCGTGATCCTGGTCATGGT
GCTGGCTATCTACTACCTGAACGCCACCGCTAAGGGACAGAAGGCCGCCAACCT
GGACCTGAAAAAGAAGATGAAAATGCAGGCCCTGGAAAACAAGATGAGAAACA
AGAAGATGGCCGCCGCTAGAGCCGCTGCCGCTGCTGGCAGACAGTGAGgatccaatca
acctctggattacaaaatttgtgaaagattgactggtattcttaactatgttgctccttttacgctatgtggatacgctgctttaatgcctttg
tatcatgctattgcttcccgtatggctttcattttctcctccttgtataaatcctggttgctgtctctttatgaggagttgtggcccgttgtca
ggcaacgtggcgtggtgtgcactgtgtttgctgacgcaacccccactggttggggcattgccaccacctgtcagctcctttccgggactttcgc
tttccccctccctattgccacggeggaactcatcgccgcctgccttgcccgctgctggacaggggctcggctgttgggcactgacaattc
cgtggtgttgtcggggaaatcatcgtcctttccttggctgctcgcctgtgttgccacctggattctgcgcgggacgtccttctgctacgtcc
cttcggccctcaatccagcggaccttccttcccgcggcctgctgccggctctgcggcctcttccgcgtcttcgACGCGTGGTA
CCTCTAGAGTCGACCCGGGCGGCCTCGAGGACGGGGTGAACTACGCCTGAGGAT
CCGATCTTTTTCCCTCTGCCAAAAATTATGGGGACATCATGAAGCCCCTTGAGCA
TCTGACTTCTGGCTAATAAAGGAAATTTATTTTCATTGCAATAGTGTGTTGGAATT
TTTTGTGTCTCTCACTCGGAAGCAATTCGTTGATCTGAATTTCGACCACCCATAAT
ACCCATTACCCTGGTAGATAAGTAGCATGGCGGGTTAATCATTAACTACAAGGA
ACCCCTAGTGATGGAGTTGGCCACTCCCTCTCTGCGCGCTCGCTCGCTCACTGAG
GCCGGGCGACCAAAGGTCGCCCGACGCCCGGGCTTTGCCCGGGCGGCCTCAGTG
AGCGAGCGAGCGCGCAG
Elements of SEQ ID NO: 10:
5′ ITR-SEQ ID NO: 6
CTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCC
AACTCCATCACTAGGGGTTCCT
CMV IE enhancer-SEQ ID NO: 17
cgttacataacttacggtaaatggcccgcctggctgaccgcccaacgacccccgcccattgacgtcaataatgacgtatgttcccatag
taacgccaatagggactttccattgacgtcaatgggtggagtatttacggtaaactgcccacttggcagtacatcaagtgtatCatatgc
caagtacgccccctattgacgtcaatgacggtaaatggcccgcctggcattatgcccagtacatgaccttatgggactttcctacttggc
agtacatctacgtattagtcatcgctattaccatg
CMV promoter-SEQ ID NO: 18
gtgatgcggttttggcagtacatcaatgggcgtggatagcggtttgactcacggggatttccaagtctccaccccattgacgtcaatggg
agtttgttttggcaccaaaatcaacgggactttccaaaatgtcgtaacaactccgccccattgacgcaaatgggcggtaggcgtgtacg
gtgggaggtctatataagcagagct
chimeric intron-SEQ ID NO: 20
gtaagtatcaaggttacaagacaggtttaaggagaccaatagaaactgggcttgtcgagacagagaagactcttgcgtttctgataggc
acctattggtcttactgacatccactttgcctttctctccacag
Weak Kozak-SEQ ID NO: 4
ccgcgg
hTMC1-opt-SEQ ID NO: 1
ATGAGCCCTAAGAAGGTGCAGATCAAGGTGGAAGAAAAGGAGGACGAGACAGA
AGAGAGCTCTTCTGAAGAGGAAGAGGAAGTGGAAGATAAGCTGCCACGGAGAG
AAAGCCTGCGCCCCAAGAGAAAGAGAACCAGAGATGTGATCAACGAGGACGAT
CCTGAACCTGAGCCTGAGGACGAGGAAACAAGAAAAGCACGAGAGAAGGAACG
GCGGAGAAGGCTGAAGCGGGGCGCTGAGGAAGAAGAAATCGACGAGGAGGAGC
TGGAAAGACTTAAGGCCGAGCTGGATGAGAAGCGCCAAATCATCGCCACAGTGA
AATGCAAGCCCTGGAAGATGGAAAAGAAAATCGAGGTGCTGAAGGAGGCCAAA
AAGTTCGTCTCCGAGAATGAGGGCGCCCTCGGTAAGGGCAAGGGCAAGCGGTGG
TTCGCCTTCAAGATGATGATGGCTAAGAAGTGGGCCAAGTTCCTGCGGGACTTCG
AGAATTTCAAGGCCGCTTGTGTGCCTTGGGAAAACAAGATTAAGGCAATCGAGA
GCCAATTTGGAAGCAGCGTGGCCTCCTACTTTCTGTTTCTGAGATGGATGTACGG
AGTGAACATGGTGCTGTTCATCCTGACATTTAGCCTGATCATGCTGCCTGAGTAC
CTATGGGGCCTGCCCTACGGCAGCCTGCCTCGGAAGACCGTGCCTCGGGCCGAA
GAGGCCAGCGCCGCCAACTTCGGCGTGCTGTACGACTTCAACGGCCTTGCTCAGT
ACAGCGTGCTGTTTTACGGCTACTACGACAACAAACGGACCATCGGCTGGATGA
ACTTCAGACTGCCTCTGAGCTACTTCCTGGTGGGCATCATGTGCATCGGATACTC
TTTCCTGGTTGTGCTCAAAGCCATGACCAAGAACATCGGCGACGACGGCGGAGG
CGATGATAACACCTTCAATTTTTCTTGGAAAGTGTTCACCAGCTGGGACTACCTG
ATTGGAAACCCTGAGACAGCCGATAATAAGTTCAATAGCATCACCATGAACTTC
AAGGAAGCCATCACCGAGGAGAAAGCCGCCCAAGTGGAAGAAAACGTTCATCT
GATCAGATTCCTGAGATTCCTGGCCAACTTTTTCGTGTTCCTGACCCTGGGCGGA
AGCGGCTACCTGATCTTCTGGGCCGTGAAGCGGAGCCAGGAGTTCGCCCAGCAG
GACCCCGACACCCTGGGATGGTGGGAAAAGAACGAGATGAACATGGTGATGTCT
CTGCTCGGCATGTTCTGTCCTACCCTGTTCGACCTGTTTGCCGAGCTGGAGGATTA
CCACCCTCTGATCGCCCTGAAGTGGCTGCTGGGTAGAATCTTCGCCCTGCTGCTC
GGCAACCTGTATGTGTTCATCCTGGCCCTGATGGATGAGATCAACAATAAGATCG
AAGAGGAAAAGCTGGTGAAGGCCAACATTACCCTGTGGGAAGCCAATATGATCA
AGGCCTATAACGCCTCCTTCAGCGAAAACAGCACCGGCCCTCCATTCTTCGTGCA
CCCCGCCGACGTGCCAAGAGGCCCTTGCTGGGAGACAATGGTAGGCCAGGAGTT
TGTTAGACTCACAGTGTCCGATGTGCTGACCACATACGTGACCATCCTGATCGGC
GACTTCCTGAGGGCCTGCTTCGTCAGATTTTGCAACTACTGCTGGTGCTGGGACC
TGGAGTACGGCTACCCCAGCTATACAGAGTTCGACATCAGCGGCAACGTCCTGG
CCCTGATCTTTAACCAGGGCATGATTTGGATGGGCAGCTTCTTCGCCCCTAGCCT
GCCTGGCATCAACATCCTGAGACTGCACACAAGCATGTACTTCCAGTGCTGGGCC
GTGATGTGTTGTAACGTGCCCGAGGCTAGAGTGTTCAAGGCCAGCAGAAGCAAC
AACTTCTACCTGGGCATGCTGCTTCTGATCCTGTTCCTGTCCACCATGCCTGTGCT
GTACATGATCGTGTCCCTGCCACCTAGCTTCGACTGCGGCCCTTTCAGTGGAAAA
AACAGAATGTTCGAGGTGATCGGCGAGACCCTGGAGCACGACTTTCCCTCTTGG
ATGGCTAAGATCCTGCGGCAGCTGTCTAATCCTGGCCTCGTGATCGCCGTGATCC
TGGTCATGGTGCTGGCTATCTACTACCTGAACGCCACCGCTAAGGGACAGAAGG
CCGCCAACCTGGACCTGAAAAAGAAGATGAAAATGCAGGCCCTGGAAAACAAG
ATGAGAAACAAGAAGATGGCCGCCGCTAGAGCCGCTGCCGCTGCTGGCAGACAG
TGA
WPRE-SEQ ID NO: 5
aatcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggcccgt
tgtcaggcaacgtggcgtggtgtgcactgtgtttgctgacgcaacccccactggttggggcattgccaccacctgtcagctcctttccgggact
ttcgctttccccctccctattgccacggcggaactcatcgccgcctgccttgcccgctgctggacaggggctcggctgttgggcactga
caattccgtggtgttgtcggggaaatcatcgtcctttccttggctgctcgcctgtgttgccacctggattctgcgcgggacgtccttctgct
acgtcccttcggccctcaatccagcggaccttccttcccgcggcctgctgccggctctgcggcctcttccgcgtcttcg
Beta-globin poly(A) signal-SEQ ID NO: 3
AATAAAGGAAATTTATTTTCATTGCAATAGTGTGTTGGAATTTTTTGTGTCTCTCA
3′ ITR-SEQ ID NO: 7
AGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTC
AGTGAGCGAGCGAGCGCGCAG
SEQ ID NO: 13
AAV9.php.b capsid protein encoding nucleic acid
CCAATGATACGCGTCGGTGCGGGCCTCTTCGCTATTACGCCAGCTGGCGAAAGG
GGGATGTGCTGCAAGGCGATTAAGTTGGGTAACGCCAGGGTTTTCCCAGTCACG
ACGTTGTAAAACGACGGCCAGTGAGCGCGCGTAATACGACTCACTATAGGGCGA
ATTGGGTACATCGACGGTATCGGGGGAGCTCGCAGGGTCTCCATTTTGAAGCGG
GAGGTTTGAACGCGCAGCCGCCATGCCGGGGTTTTACGAGATTGTGATTAAGGTC
CCCAGCGAC
CTTGACGAGCATCTGCCCGGCATTTCTGACAGCTTTGTGAACTGGGTGGCCGAGA
AGGAATGGGAGTTGCCGCCAGATTCTGACATGGATCTGAATCTGATTGAGCAGG
CACCCCTGACCGTGGCCGAGAAGCTGCAGCGCGACTTTCTGACGGAATGGCGCC
GTGTGAGTAAGGCCCCGGAGGCTCTTTTCTTTGTGCAATTTGAGAAGGGAGAGAG
CTACTTCCACATGCACGTGCTCGTGGAAACCACCGGGGTGAAATCCATGGTTTTG
GGACGTT
TCCTGAGTCAGATTCGCGAAAAACTGATTCAGAGAATTTACCGCGGGATCGAGC
CGACTTTGCCAAACTGGTTCGCGGTCACAAAGACCAGAAATGGCGCCGGAGGCG
GGAACAAGGTGGTGGATGAGTGCTACATCCCCAATTACTTGCTCCCCAAAACCC
AGCCTGAGCTCCAGTGGGCGTGGACTAATATGGAACAGTATTTAAGCGCCTGTTT
GAATCTCACGGAGCGTAAACGGTTGGTGGCGCAGCATCTGACGCACGTGTCGCA
GACGCAGGA G C AGAAC AAAGAGAAT C AGAAT C C C AAT T C T GAT
GCGCCGGT GAT C AGAT C
AAAAACTTCAGCCAGGTACATGGAGCTGGTCGGGTGGCTCGTGGACAAGGGGAT
TACCTCGGAGAAGCAGTGGATCCAGGAGGACCAGGCCTCATACATCTCCTTCAA
TGCGGCCTCCAACTCGCGGTCCCAAATCAAGGCTGCCTTGGACAATGCGGG
AAAGATTATGAGCCTGACTAAAACCGCCCCCGACTACCTGGTGGGCCAGCAGCC
CGTGGAGGACATTTCCAGCAATCGGATTTATAAAATTTTGGAACTAAACGGGTAC
GATCCCCAATATGCGGCTTCCGTCTTTCTGGGATGGGCCACGAAAAAGTTCGGCA
AGAGGAACACCATCTGGCTGTTTGGGCCTGCAACTACCGGGAAGACCAACATCG
CGGAGGCCATAGCCCACACTGTGCCCTTCTACGGGTGCGTAAACTGGACCAATG
AGAACTTT
CCCTTCAACGACTGTGTGGACAAGATGGTGATCTGGTGGGAGGAGGGGAAGATG
ACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTC
ACCTCCAACACCAATATGTGCGCCGTGATTGACGGGAACTCAACGACCTTCGAA
CACCAGCAGCCGTTGCAAGACCGGATGTTCAAATTTGAACTCACCCGCCGTCTGG
ATCATGACT
TTGGGAAGGTCACCAAGCAGGAAGTCAAAGACTTTTTCCGGTGGGCAAAGGATC
ACGTGGTTGAGGTGGAGCATGAATTCTACGTCAAAAAGGGTGGAGCCAAGAAAA
GACCCGCCCCCAGTGACGCAGATATAAGTGAGCCCAAACGGGTGCGCGAGTCAG
TTGCGCAGCCATCGACGTCAGACGCGGAAGCTTCGATCAACTACGCGGACAGGT
ACCAAAACAAATGTTCTCGTCACGTGGGCATGAATCTGATGCTGTTTCCCTGCAG
ACAATGCGA
GAGACTGAATCAGAATTCAAATATCTGCTTCACTCACGGTGTCAAAGACTGTTTA
GAGTGCTTTCCCGTGTCAGAATCTCAACCCGTTTCTGTCGTCAAAAAGGCGTATC
AGAAACTGTGCTACATTCATCACATCATGGGAAAGGTGCCAGACGCTTGCACTG
CTTGCGACCTGGTCAATGTGGACTTGGATGACTGTGTTTCTGAACAATAAATGAC
TTAAACCAGGTATGAGTCGGCTGGATAAATCTAAAGTCATAAACGGCGCTCTGG
AATTACTCAATGAAGTCGGTATCGAAGGCCTGACGACAAGGAAACTCGCTCAAA
AGCTGGGAGTTGAGCAGCCTACCCTGTACTGGCACGTGAAGAACAAGCGGGCCC
TGCTCGATGCCCTGGCCATCGAGATGCTGGACAGGCATCATACCCACTTCTGCCC
CCTGGAAGGCGAGTCATGGCAAGACTTTCTGCGGAACAACGCCAAGTCATTCCG
CTGTGCTCTCCTCTCACATCGCGACGGGGCTAAAGTGCATCTCGGCACCCGCCCA
ACAGAGAAACAGTACGAAACCCTGGAAAATCAGCTCGCGTTCCTGTGTCAGCAA
GGCTTCTCCCTGGAGAACGCACTGTACGCTCTGTCCGCCGTGGGCCACTTTACAC
TGGGCTGCGTATTGGAGGAACAGGAGCATCAAGTAGCAAAAGAGGA
AAGAGAGAC AC C T AC C AC C GAT T C TAT G CC C
CCACTTCTGAGACAAGCAATTGAGCTGTTCGACCGGCAGGGAGCCGAACCTGCC
TTCCTTTTCGGCCTGGAACTAATCATATGTGGCCTGGAGAAACAGCTAAAGTGCG
AAAGCGGCGGGCCGGCCGACGCCCTTGACGATTTTGACTTAGACATGCTCCCAG
CCGATGCCCTTGACGACTTTGACCTTGATATGCTGCCTGCTGACGCTCTTGACGA
TTTTGACCTTGACATGCTCCCCGGGTAAATGCATGAATTCGATCTAGAGGGCCCT
ATTCTATAGTGTCACCTAAATGCTAGAGCTCGCTGATCAGCCTCGACTGTGCCTT
CTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTC
TGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTT
CTGAGGCGGAAAGAACCAGCTGGGGCTCGAATCAAGCTATCAAGTGCCACCTGA
CGTCTCCCTATCAGTGATAGAGAAGTCGACACGTCTCGAGCTCCCTATCAGTGAT
AGAGAAGGTACGTCTAGAACGTCTCCCTATCAGTGATAGAGAAGTCGACACGTC
TCGAGCTCCCTATCAGTGATAGAGAAGGT
ACGTCTAGAACGTCTCCCTATCAGTGATAGAGAAGTCGACACGTCTCGAGCTCCC
TATCAGTGATAGAGAAGGTACGTCTAGAACGTCTCCCTATCAGTGATAGAGAAG
TCGACACGTCTCGAGCTCCCTATCAGTGATA GAGAAG GT AC C C C C TAT AT
AAG CAGAGAGAT C T GT T C AAAT T T GAAC T GAC T AAG CGGCTCCCGC C
AGAT
TTTGGCAAGATTACTAAGCAGGAAGTCAAGGACTTTTTTGCTTGGGCAAAGGTCA
ATCAGGTGCCGGTGACTCACGAGTTTAAAGTTCCCAGGGAATTGGCGGGAACTA
AAGGGGCGGAGAAATCTCTAAAACGCCCACTGGGTGACGTCACCAATACTAGCT
ATAAAAGTCTGGAGAAGCGGGCCAGGCTCTCATTTGTTCCCGAGACGCCTCGCA
GTTCAGACGTGACTGTTGATCCCGCTCCTCTGCGACCGCTAGCTTCGATCAACTA
CGCAGACA
GGTACCAAAACAAGTGTTCTCGTCACGTGGGCATTAATCTGATTCTGTTTCCCTG
CAGACAATGCGAGAGAATGAATCAGAACTCAAATATCTGCTTCACTCACGGACA
GAAAGACTGTTTAGAGTGCTTTCCCGTGTCAGAATCTCAACCCGTTTCTGTCGTC
AAAAAGGCGTATCAGAAACTGTGCTACATTCATCATATCATGGGAAAGGTGCCA
GACGCTTGCACTGCCTGCGATCTGGTCAATGTGGATTTGGATGACTGCATCTTTG
AACAATA
AATGACTTAAGCCAGGTATGGCTGCCGATGGTTATCTTCCAGATTGGCTCGAGGA
CAACCTTAGTGAAGGAATTCGCGAGTGGTGGGCTTTGAAACCTGGAGCCCCTCA
ACCCAAGGCAAATCAACAACATCAAGACAACGCTAGAGGTCTTGTGCTTCCGGG
TTACAAATACCTTGGACCCGGCAACGGACTCGACAAGGGGGAGCCGGTCAACGC
AGCAGACGCGGCGGCCCTCGAGCACGACAAAGCCTACGACCAGCAGCTCAAGGC
CGGAGACAA
CCCGTACCTCAAGTACAACCACGCCGACGCCGAGTTCCAGGAGCGGCTCAAAGA
AGATACGTCTTTTGGGGGCAACCTCGGGCGAGCAGTCTTCCAGGCCAAAAAGAG
GCTTCTTGAACCTCTTGGTCTGGTTGAGGAAGCGGCTAAGACGGCTCCTGGAAAG
AAGAGGCCTGTAGAGCAGTCTCCTCAGGAACCGGACTCCTCCGCGGG TAT T G G
C AAAT C G G GT G C AC AG C C C GC T AAAAAGAGAC T C AAT T T C G GT C
AGACTGG C
GACACAGAGTCAGTCCCAGACCCTCAACCAATCGGAGAACCTCCCGCAGCCCCC
TCAGGTGTGGGATCTCTTACAATGGCTTCAGGTGGTGGCGCACCAGTGGCAGAC
AATAACGAAGGTGCCGATGGAGTGGGTAGTTCCTCGGGAAATTGGCATTGCGAT
TCCCAATGGCTGGGGGACAGAGTCATCACCACCAGCACCCGAACCTGGGCCCTG
CCCACC T AC AAC AAT C AC C T C T AC AAG C AAAT C T C C AAC AG C AC AT
CTG GAG
GATCTTCAAATGACAACGCCTACTTCGGCTACAGCACCCCCTGGGGGTATTTTGA
CTTCAACAGATTCCACTGCCACTTCTCACCACGTGACTGGCAGCGACTCATCAAC
AACAACTGGGGATTCCGGCCTAAGCGACTCAACTTCAAGCTCTTTAACATTCAGG
TCAAAGAGGTTACGGACAACAATGGAGTCAAGACCATCGCCAATAACCTTACCA
GCACGGTCCAGGTCTTCACGGACTCAGACTATCAGCTCCCGTACGTGCTCGGGTC
GGCTCACGAGGGCTGCCTCCCGCCGTTCCCAGCGGACGTTTTCATGATTCCTCAG
TACGGGTATCTGACGCTTAATGATGGAAGCCAGGCCGTGGGTCGTTCGTCCTTTT
ACTGCCTGGAATATTTCCCGTCGCAAATGCTAAGAACGGGTAACAACTTCCAGTT
CAGCT AC GAGT T T GAGAAC GTACCTTTC CAT AG C AG C T AC G C T C AC AG
C C AAAG C CT G GAC C GAC T AAT GAAT C C ACT CAT C GAC C AAT AC TT
GT AC TAT CT C T C T AGAAC TAT T AAC G GT T C T G GAC AGAAT C AAC
AAACGCT
AAAATTCAGTGTGGCCGGACCCAGCAACATGGCTGTCCAGGGAAGAAACTACAT
ACCTGGACCCAGCTACCGACAACAACGTGTCTCAACCACTGTGACTCAAAACAA
CAACAGCGAATTTGCTTGGCCTGGAGCTTCTTCTTGGGCTCTCAATGGACGTAAT
AGCTTGATGAATCCTGGACCTGCTATGGCCTCTCACAAAGAAGGAGAGGACCGT
TTCTTTCCTTTGTCTGGATCTTTAATTTTTGGCAAACAAGGTACTGGCAGAGACAA
CGTGGATG C G GAC AAAGT CAT GAT AAC C AAC GAAGAAGAAAT T AAAAC T
ACTAAC CC G
GTAGCAACGGAGTCCTATGGACAAGTGGCCACAAACCACCAGAGTGCCCAAACT
TTGGCGGTGCCTTTTAAGGCACAGGCGCAGACCGGTTGGGTTCAAAACCAAGGA
ATACTTCCGGGTATGGTTTGGCAGGACAGAGATGTGTACCTGCAAGGACCCATTT
GGGCCAAAATTCCTCACACGGACGGCAACTTTCACCCTTCTCCGCTGATGGGAGG
GTTTGGAATGAAGCACCCGCCTCCTCAGATCCTCATCAAAAACACACCTGTACCT
GCGGATCCTCCAACGGCCTTCAACAAGGACAAGCTGAACTCTTTCATCACCCAGT
ATTCTACTGGTCAAGTCAGCGTGGAGATCGAGTGGGAGCTGC AGAAG GAAAAC
AG CAAGCGCTG
GAACCCGGAGATCCAGTACACTTCCAACTATTACAAGTCTAATAATGTTGAATTT
GCTGTTAATACTGAAGGTGTATATAGTGAACCCCGCCCCATTGGCACCAGATACC
TGACTCGTAATCTGTAAGTCGACTTGCTTGTTAATCAATAAACCGTTTAATTCGTT
TCAGTTGAACTTTGGTCTCTGCGAAGGGCAATTCGTTTAAACCTGCAGGACTAGA
GGTCCTGTATTAGAGGTCACGTGAGTGTTTTGCGACATTTTGCGACACCATGTGG
TCACGCTGGGTATTTAAGCCCGAGTGAGCACGCAGGGTCTCCATTTTGAAGCGGG
AGGTTTGAACGCGCAGCCGCCAAGCCGAATTCTGCAGATATCACATGTCCTAGG
AACTATCGATCCA
TCACACTGGCGGCCGCTCGACTAGAGCGGCCGCCACCGCGGTGGAGCTCCAGCT
TTTGCGGACCGAATCGGAAAGAACATGTGAGCAAAAGGCCAGCAAAAGGCCAG
GAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTA
TAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTC
TGCTGAAGCCAGTTACCTTCGGAAAAAGAGTTGGTAGCTCTTGATCCGGCAAAC
AAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAG
TGGAA C GAAAAC T C AC GT T AAG G GAT T T T G GT CAT GAGAT TAT C
AAAAAG GAT C T T C AC C
TAGATCCTTTTAAATTAAAAATGAAGTTTTAAATCAATCTAAAGTATATATGAGT
AAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGA
TACGGGAGGGCTTACCATCTGGCCCCAGTGCTGCAATGATACCGCGAGACCCAC
GCTCA
CCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAG
CTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCC
AACGATCAAGGCGAGTTACATGATCCCCCATGTTGTGCAAAAAAGCGGTTAGCT
CCTTCG
GTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTG
CTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCG
CTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGC
AAAAAAGGGAATAAGGGCGACACGGAAATGTTGAA TACT CAT AC
TCTTCCTTTTT C AAT AT TAT T
GAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGA
C GTC
SEQ ID NO: 14-AAV Anc80 capsid protein amino acid sequence
MAADGYLPDWLEDNLSEGIREWWDLKPGAPKPKANQQKQDDGRGLVLPGYKYLGPFNGLDKGEPV
NAADAAALEHDKAYDQQLKAGDNPYLRYNHADAEFQERLQEDTSFGGNLGRAVFQAKKRVLEPLG
LVEEGAKTAPGKKRPVEQSPQEPDSSSGIGKKGQQPAX₁KRLNFGQTGDSESVPDPQPLGEPPAAP
SGVGSNTMX₂AGGGAPMADNNEGADGVGNASGNWHCDSTWLGDRVITTSTRTWALPTYNNHLYKQI
SSQSGX₃STNDNTYFGYSTPWGYFDFNRFHCHFSPRDWQRLINNNWGFRPKX₄LNFKLFNIQVKEV
TTNDGTTTIANNLTSTVQVFTDSEYQLPYVLGSAHOGCLPPFPADVFMIPQYGYLTLNNGSQAVG
RSSFYCLEYFPSQMLRTGNNFX₅FSYTFEDVPFHSSYAHSQSLDRLMNPLIDQYLYYLSRTQTTSG
TAGNRX₆LQFSQAGPSSMANQAKNWLPGPCYRQQRVSKTX₇NQNNNSNFAWTGATKYHLNGRDSLV
NPGPAMATHKDDEDKFFPMSGVLIFGKQGAGNSNVDLDNVMITX₈EEEIKTTNPVATEX₉YGTVAT
NLQSX₁₀NTAPATGTVNSQGALPGMVWQX₁₁RDVYLQGPIWAKIPHTDGHFHPSPLMGGFGLKHPPP
QILIKNTPVPANPPTTFSPAKFASFITQYSTGQVSVEIEWELQKENSKRWNPEIQYTSNYNKSTN
VDFAVDTNGVYSEPRPIGTRYLTRNL
where X₁ = K/R; X₂ = A/S; X₃ = A/G; X₄ = R/K; X₅ = E/Q; X₆ = T/E; X₇ = A/T;
X₈ = S/N; X₉ = Q/E; X₁₀ = S/A; X₁₁ = N/D
SEQ ID NO: 15-AAV Anc80 capsid protein amino acid sequence
MAADGYLPDWLEDNLSEGIREWWDLKPGAPKPKANQQKQDDGRGLVLPGYKYLGPFNGLDKGEPV
NAADAAALEHDKAYDQQLKAGDNPYLRYNHADAEFQERLQEDTSFGGNLGRAVFQAKKRVLEPLG
LVEEGAKTAPGKKRPVEQSPQEPDSSSGIGKKGQQPARKRLNFGQTGDSESVPDPQPLGEPPAAP
SGVGSNTMAAGGGAPMADNNEGADGVGNASGNWHCDSTWLGDRVITTSTRTWALPTYNNHLYKQI
SSQSGGSTNDNTYFGYSTPWGYFDFNRFHCHFSPRDWQRLINNNWGFRPKKLNFKLFNIQVKEVT
TNDGTTTIANNLTSTVQVFTDSEYQLPYVLGSAHQGCLPPFPADVFMIPQYGYLTLNNGSQAVGR
SSFYCLEYFPSQMLRTGNNFQFSYTFEDVPFHSSYAHSQSLDRLMNPLIDQYLYYLSRTQTTSGT
AGNRTLQFSQAGPSSMANQAKNWLPGPCYRQQRVSKTTNQNNNSNFAWTGATKYHLNGRDSLVNP
GPAMATHKDDEDKFFPMSGVLIFGKQGAGNSNVDLDNVMITNEEEIKTTNPVATEEYGTVATNLQ
SANTAPATGTVNSQGALPGMVWQDRDVYLQGPIWAKIPHTDGHFHPSPLMGGFGLKHPPPQILIK
NTPVPANPPTTFSPAKFASFITQYSTGQVSVEIEWELQKENSKRWNPEIQYTSNYNKSTNVDFAV
DTNGVYSEPRPIGTRYLTRNL

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Unless the context indicates otherwise, it is specifically intended that the various features described herein can be used in any combination.

### EXAMPLES

### Example 1: Presence of Woodchuck Hepatitis Virus Posttranscriptional Regulatory Element (WPRE) in the AAV Expression Cassette Expressing TMC1 Promotes More Durable Hearing Recovery

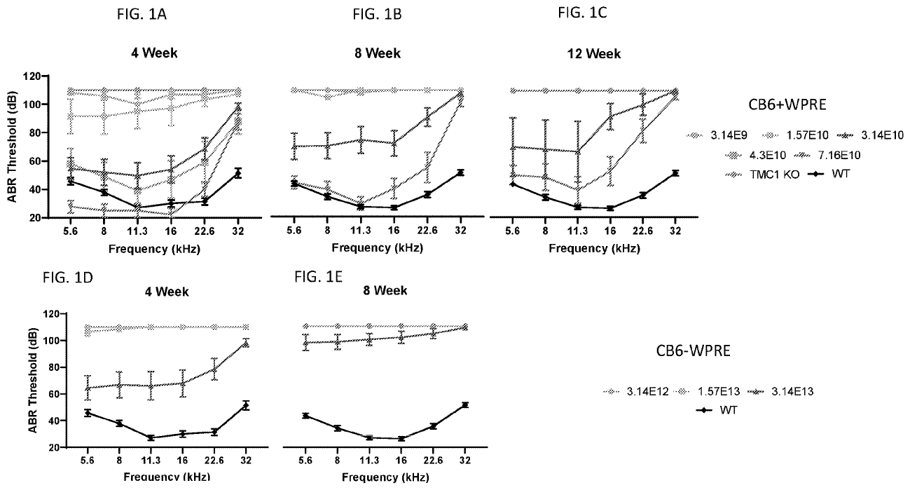

TMC1 knockout mice were administered via intracochlear injection, different doses (vector genomes per cochlea) of a recombinant adeno-associated vector (rAAV), comprising an AAV expression cassette having the nucleic acid sequence of SEQ ID NO: 8 (comprising a 5′ITR, a CB6 promoter, a Kozak sequence, a transgene encoding TMC1, a WPRE, a beta globin poly A sequence, and a 3′ ITR) or SEQ ID NO: 9 (comprising a 5′ ITR, a CB6 promoter, a Kozak sequence, a transgene encoding TMC1, a beta globin poly A sequence, and a 3′ ITR), and the auditory brain stem response (ABR) threshold was measured at different time points (such as, 4 weeks, 8 weeks, or 12 weeks after administration) at different frequencies. Wildtype mice (WT) and TMC1 knockout (TMC1 KO) mice were used as controls. As shown in FIGS. 1A-1E, TMC1 KO mice that were injected with an AAV expression cassette of SEQ ID NO: 8 (comprising a WPRE) exhibited a more durable and more improved hearing recovery (FIGS. 1A-1C), as compared to mice that were injected with an AAV expression cassette of SEQ ID NO: 9 (lacking a WPRE) (FIGS. 1D-1E).

The results show that the expression of TMC1 from an expression cassette containing WPRE results in more durable and improved recovery of the hearing function, as compared to the expression of TMC1 from an expression cassette lacking WPRE. Therefore, the presence of WPRE in the expression cassette has an unexpected effect on the rescue of the hearing function.

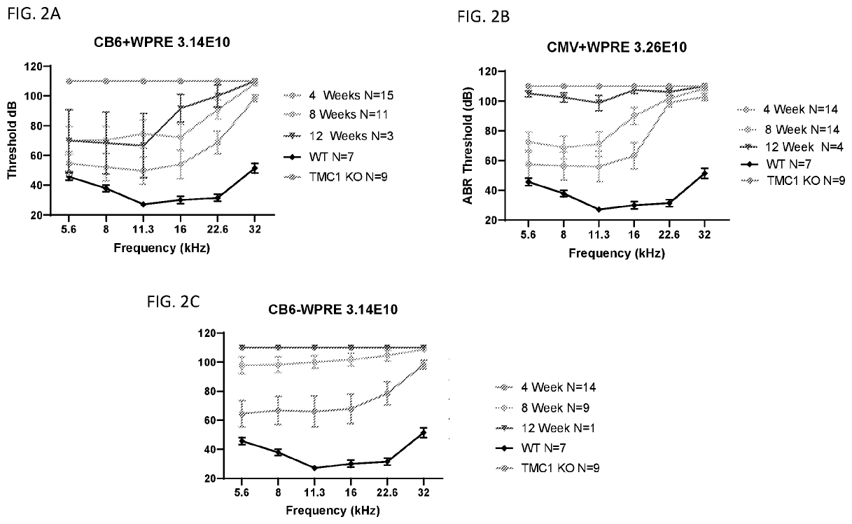

This surprising effect is also observed when the same dosage of rAAV is injected into the mice. For instance, FIGS. 2A and 2C show that TMC1 KO mice that were injected with 3.14E10 vector genomes/cochlea of AAV expression cassette of SEQ ID NO: 8 (comprising a WPRE) exhibited a more durable and more improved hearing recovery, as compared to mice that were injected with the same dose of an AAV expression cassette of SEQ ID NO: 9 (lacking a WPRE).

### Example 2: Presence of CB6 Promoter in the AAV Expression Cassette Expressing TMC1 Promotes More Durable Hearing Recovery

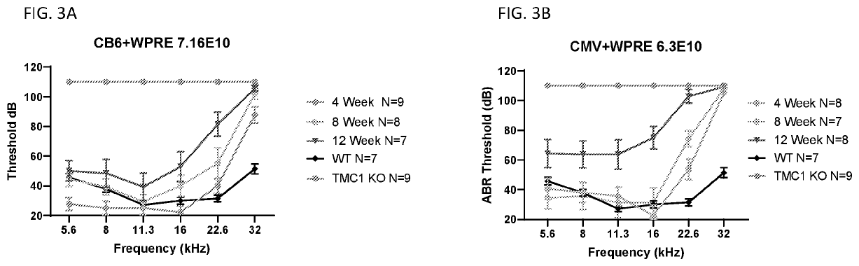

TMC1 knockout mice were administered via intracochlear injection, a recombinant adeno-associated vector (rAAV), comprising an AAV expression cassette having the nucleic acid sequence of SEQ ID NO: 8 (comprising a 5′ITR, a CB6 promoter, a Kozak sequence, a transgene encoding TMC1, a WPRE, a beta globin polyA sequence, and a 3′ ITR) or SEQ ID NO: 10 (comprising a 5′ITR, a CMV promoter, a Kozak sequence, a transgene encoding TMC1, a WPRE, a beta globin polyA sequence, and a 3′ ITR), and the auditory brain stem response (ABR) threshold was measured at different time points (such as, 4 weeks, 8 weeks, or 12 weeks after administration) at different frequencies. Wildtype mice (WT) and TMC1 knockout (TMC1 KO) mice were used as controls. As shown in FIGS. 2A-2B and 3A-3B, TMC1 KO mice that were injected with an AAV expression cassette of SEQ ID NO: 8 (comprising a CB6 promoter) exhibited a more durable and more improved hearing recovery, as compared to mice that were injected with an AAV expression cassette of SEQ ID NO: 10 (comprising a CMV promoter). These results show that expressing TMC1 using the CB6 promoter disclosed herein has an unexpected effect on the rescue of the hearing function.

### Example 3: Expression of TMC1 Using AAV-Mediated Gene Therapy Promotes Hair Cell Survival

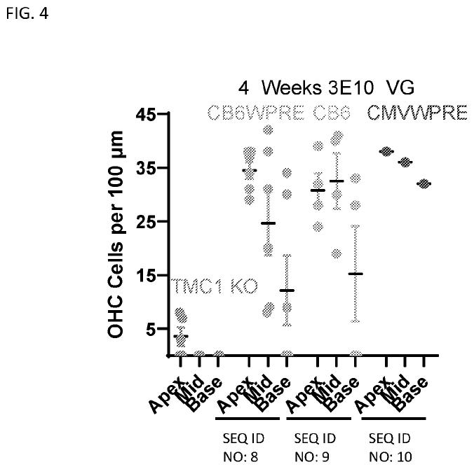

TMC1 knockout mice were administered via intracochlear injection, the same dosage (3E10 vector genome/cochlea) of a recombinant adeno-associated vector (rAAV), comprising an AAV expression cassette having the nucleic acid sequence of SEQ ID NO: 8 (comprising a WPRE element and a CB6 promoter), SEQ ID NO: 9 (lacking a WPRE element), or SEQ ID NO: 10 (comprising a CMV promoter), and the number of outer hair cells were evaluated at 4 weeks post administration. TMC1 knockout (TMC1 KO) mice were used as control. As shown in FIG. 4, expression of TMC1 from SEQ ID NO: 8, 9, or 10 resulted in a remarkable increase in the number of outer hair cells (OHC), as compared to TMC1 KO mice. These results show that expression of TMC1 using AAV-based gene therapy disclosed herein improves hearing at least in part by promoting the survival of hair cells.

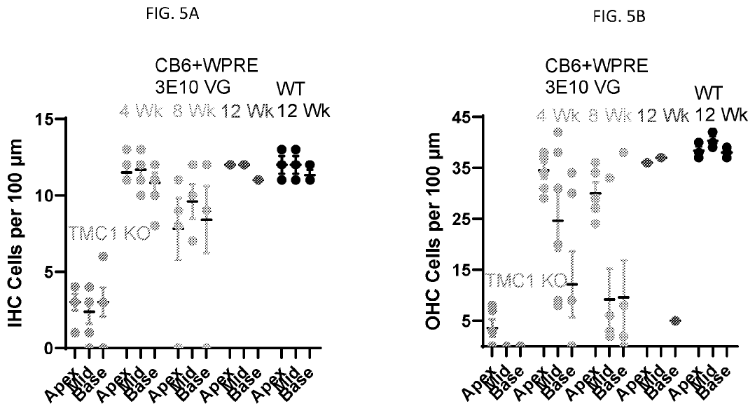

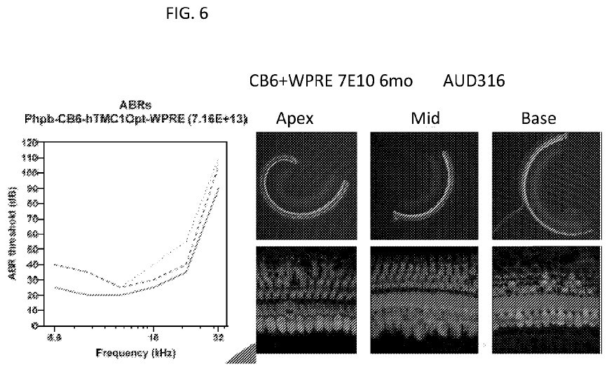

The effect of TMC1 expression on hair cell survival was further evaluated over time. As shown in FIGS. 5A-5B, expression of TMC1 from SEQ ID NO: 8 resulted in a remarkably durable increase in the number of outer hair cells (OHC) as well as inner hair cells (IHC) through 12 weeks, as compared to TMC1 KO mice. These results show that expression of TMC1 using AAV-based gene therapy disclosed herein improves hearing, at least in part, by promoting the survival of hair cells. FIG. 6 shows the morphology of inner hair cells at 6 months after administration of a recombinant adeno-associated vector (rAAV), comprising an AAV expression cassette having the nucleic acid sequence of SEQ ID NO: 8 (comprising a WPRE element and a CB6 promoter).

### Example 4: Expression of TMC1 Using AAV-Mediated Gene Therapy Rescues the Phenotypes of N193I Mutant Mice

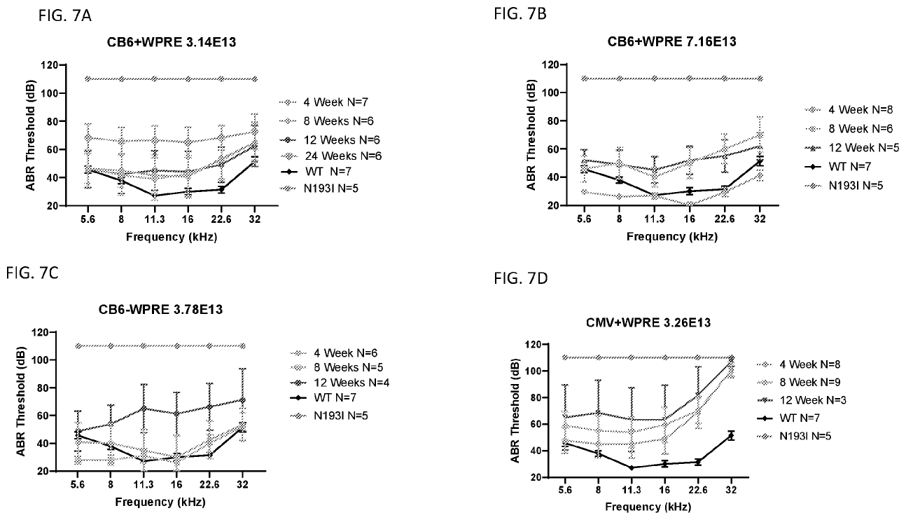

Mice carrying the “Tmc1 p.N193I mutation” are referred to herein as “N193I mutant mice” and were used in this experiment. The Tmc1 p.N193I mutation is equivalent to the hypofunctional human TMC1 mutation p.N199I (c.596A>T), which causes progressive moderate-to-severe hearing loss during childhood. N193I mutant mice were administered via intracochlear injection, different doses (vector genomes per cochlea) of a recombinant adeno-associated vector (rAAV), comprising an AAV expression cassette having the nucleic acid sequence of SEQ ID NO: 8 (comprising a WPRE) or SEQ ID NO: 9 (lacking a WPRE), and the auditory brain stem response (ABR) threshold was measured at different time points (such as, 4 weeks, 8 weeks, or 12 weeks after administration) at different frequencies. Wildtype mice (WT) and N193I mutant (N1311) mice were used as controls. As shown in FIGS. 7A and 7B, N193I mutant mice that were injected with two different doses of AAV expression cassette of SEQ ID NO: 8 (comprising a WPRE) exhibited a remarkably durable hearing recovery for as long as 24 weeks, as compared to N193I mutant mice that were injected with an AAV expression cassette of SEQ ID NO: 9 (lacking a WPRE; FIG. 7C) or SEQ ID NO: 10 (comprising a CMV promoter; FIG. 7D).

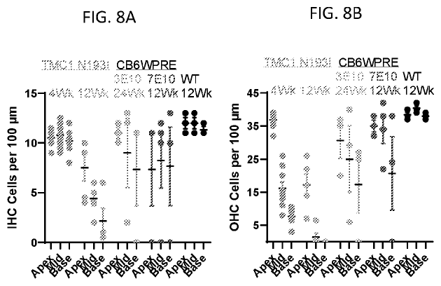

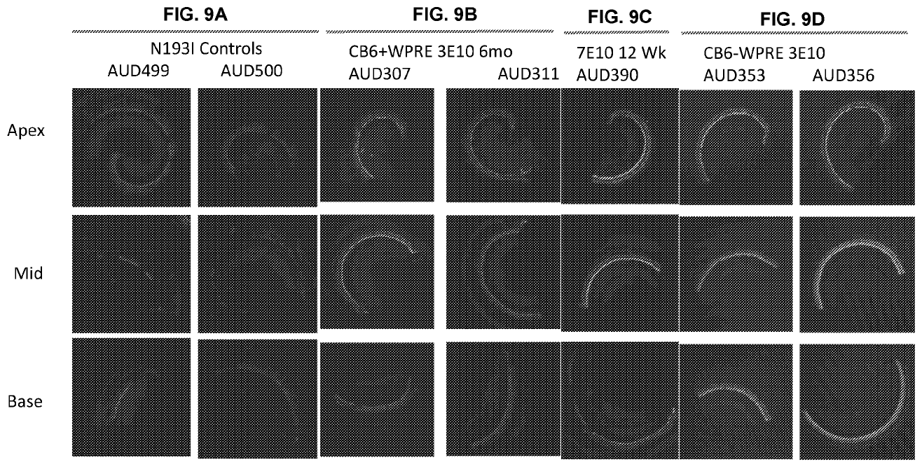

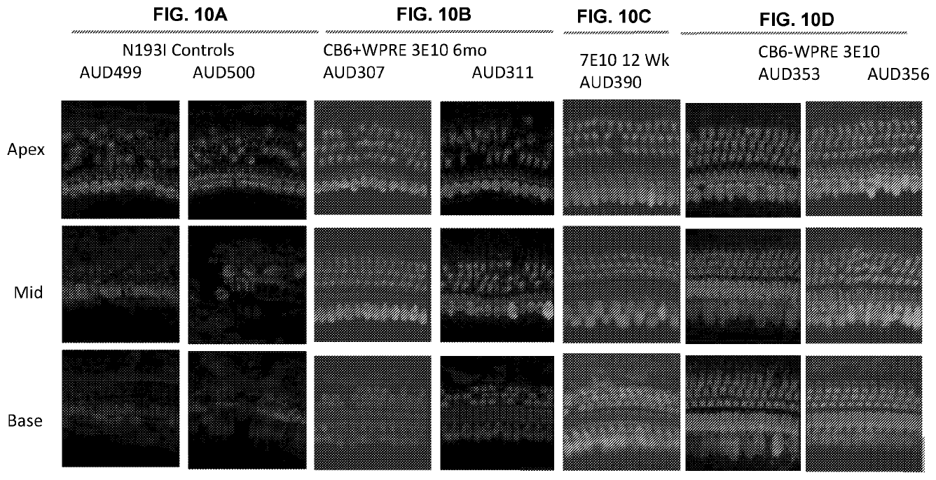

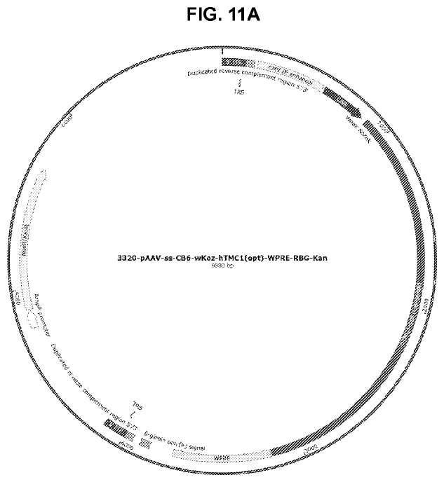

Furthermore, the effect of TMC1 expression on hair cell survival in N193I mutant mice was further evaluated over time. Wildtype (WT) and N193I mutant mice were used as controls. As shown in FIGS. 8A-8B, expression of TMC1 from SEQ ID NO: 8 resulted in a remarkably durable increase in the number of outer hair cells (OHC; FIG. 8B) as well as inner hair cells (IHC; FIG. 8A) through 12 weeks, as compared to TMC1 KO mice. These results show that expression of TMC1 using AAV-based gene therapy disclosed herein improves hearing, at least in part, by promoting the survival of hair cells. FIGS. 9A-9D and 10A-10D show the morphology of inner hair cells after administration of a recombinant adeno-associated vector (rAAV), comprising an AAV expression cassette having the nucleic acid sequence of SEQ ID NO: 8 (comprising a WPRE element and a CB6 promoter) or SEQ ID NO: 9 (lacking WPRE).

### Numbered Embodiments

Embodiment 1. A nucleic acid molecule, comprising an adeno-associated virus (AAV) expression cassette, wherein the AAV expression cassette comprises, from 5′ to 3′:
i. a 5′ AAV inverted terminal repeat (ITR);
ii. a promoter comprising a CB6 promoter;
iii. a transgene encoding transmembrane channel-like 1 (TMC1); and
iv. a 3′ AAV ITR.

Embodiment 2. The nucleic acid molecule of claim 1, wherein the transgene encodes a human transmembrane channel-like 1 (hTMC1).

Embodiment 3. The nucleic acid molecule of claim 1 or claim 2, wherein the transgene encodes a codon-optimized human transmembrane channel-like 1 (co-hTMC1).

Embodiment 4. The nucleic acid molecule of any one of claims 1-3, wherein the transgene comprises a nucleic acid sequence having at least 90% identity to SEQ ID NO: 1.

Embodiment 5. The nucleic acid molecule of any one of claims 1-4, wherein the transgene comprises the nucleic acid sequence of SEQ ID NO: 1.

Embodiment 6. The nucleic acid molecule of any one of claims 1-5, wherein the promoter is operably linked to the transgene.

Embodiment 7. The nucleic acid molecule of any one of claims 1-6, wherein the CB6 promoter comprises the nucleic acid sequence of SEQ ID NO: 2.

Embodiment 8. The nucleic acid molecule of any one of claims 1-7 wherein the AAV expression cassette comprises a beta globin polyadenylation sequence.

Embodiment 9. The nucleic acid molecule of claim 8, wherein the beta globin polyadenylation sequence comprises the nucleic acid sequence of SEQ ID NO: 3.

Embodiment 10. The nucleic acid molecule of any one of claims 1-9, wherein the AAV expression cassette comprises a Kozak sequence.

Embodiment 11. The nucleic acid molecule of claim 10, wherein the Kozak sequence comprises the nucleic acid sequence of SEQ ID NO: 4.

Embodiment 12. The nucleic acid molecule of any one of claims 1-11, wherein the AAV expression cassette comprises a woodchuck hepatitis virus posttranscriptional regulatory element (WPRE).

Embodiment 13. The nucleic acid molecule of claim 12, wherein the WPRE comprises the nucleic acid sequence of SEQ ID NO: 5.

Embodiment 14. The nucleic acid molecule of any one of claims 1-13, wherein the 5′ AAV ITR sequence comprises the nucleic acid sequence of SEQ ID NO: 6.

Embodiment 15. The nucleic acid molecule of any one of claims 1-14, wherein the 3′ AAV ITR sequence comprises the nucleic acid sequence of SEQ ID NO: 7.

Embodiment 16. The nucleic acid molecule of any one of claims 1-15, wherein the AAV expression cassette comprises, from 5′ to 3′:
i. a 5′ AAV ITR,
ii. a CB6 promoter,
iii. a Kozak sequence,
iv. a codon-optimized human transmembrane channel-like 1 (co-hTMC1),
v. a woodchuck hepatitis virus posttranscriptional regulatory element (WPRE),
vi. a beta globin polyadenylation sequence, and
vii. a 3′ AAV ITR.

Embodiment 17. The nucleic acid molecule of any one of claims 1-16, wherein the AAV expression cassette comprises the nucleic acid sequence of SEQ ID NO: 8.

Embodiment 18. A plasmid, comprising the nucleic acid molecule of any one of claims 1-17.

Embodiment 19. A cell, comprising the nucleic acid molecule of any one of claims 1-17, or the plasmid of claim 18.

Embodiment 20. A method of producing a recombinant adeno-associated virus (rAAV), the method comprising:
i. contacting an AAV producer cell with the nucleic acid molecule of any one of claims 1-17, or the plasmid of claim 18.

Embodiment 21. A recombinant adeno-associated virus (rAAV) produced by the method of claim 20.

Embodiment 22. The rAAV of claim 21, wherein the rAAV comprises an AAV9-php.b capsid protein.

Embodiment 23. The rAAV of claim 21, wherein the rAAV comprises a AAV9 capsid protein, comprising an insertion of the amino acid sequence of SEQ ID NO: 11 (TLAVPFK) between amino acid 588 and amino acid 589, wherein the amino acids are numbered according to VP1 capsid protein.

Embodiment 24. A recombinant adeno-associated virus (rAAV), comprising:
i. an AAV9-php.b capsid protein; and the nucleic acid molecule of any one of claims 1-17.

Embodiment 25. A recombinant adeno-associated virus (rAAV), comprising: an AAV9-php.b capsid protein; and a nucleic acid molecule comprising an AAV expression cassette, wherein the AAV expression cassette comprises, from 5′ to 3′:
i. a 5′ AAV ITR comprising the nucleic acid sequence of SEQ ID NO: 6,
ii. a CB6 promoter comprising the nucleic acid sequence of SEQ ID NO: 2,
iii. a Kozak sequence comprising the nucleic acid sequence of SEQ ID NO: 4,
iv. a transgene, encoding a codon-optimized human transmembrane channel-like 1 (co-hTMC1), comprising the nucleic acid sequence of SEQ ID NO: 1,
v. a woodchuck hepatitis virus posttranscriptional regulatory element (WPRE) comprising the nucleic acid sequence of SEQ ID NO: 5,
vi. a beta globin polyadenylation sequence comprising the nucleic acid sequence of SEQ ID NO: 3, and
vii. a 3′ AAV ITR comprising the nucleic acid sequence of SEQ ID NO: 7.

Embodiment 26. A recombinant adeno-associated virus (rAAV), comprising:
i. an AAV9-php.b capsid protein; and
ii. a nucleic acid molecule, comprising an AAV expression cassette, wherein the AAV expression cassette comprises the nucleic acid sequence of SEQ ID NO: 8.

Embodiment 27. The rAAV of any one of claims 22-26, wherein the AAV9.php.b capsid protein is encoded by a nucleic acid having at least 90% identity to SEQ ID NO: 13.

Embodiment 28. The rAAV of any one of claims 22-27, wherein the AAV expression cassette comprises an miR-1 binding site, an miR-133a binding site, and/or an miR-122 binding site.

Embodiment 29. The rAAV of any one of claims 22-28, wherein the rAAV is a self-complementary AAV.

Embodiment 30. The rAAV of any one of claims 22-28, wherein the rAAV is a single-stranded AAV.

Embodiment 31. A pharmaceutical composition, comprising:
(a) the nucleic acid molecule of any one of claims 1-17, the plasmid of claim 18, the cell of claim 19, or the rAAV of any one of claims 21-30; and
(b) a pharmaceutically acceptable carrier.

Embodiment 32. A method of expressing transmembrane channel-like 1 (TMC1) in a cell, comprising:
i. contacting the cell with the nucleic acid molecule of any one of claims 1-17, the plasmid of claim 18, the cell of claim 19, or the rAAV of any one of claims 21-30, or the composition of claim 31, thereby expressing TMC1 in the cell.

Embodiment 33. The method of claim 32, wherein the contacting step is performed in vitro, ex vivo, or in vivo.

Embodiment 34. The method of claim 33, wherein the contacting step is performed in vivo in a subject in need thereof.

Embodiment 35. The method of claim 34, wherein the contacting step comprises administering a therapeutically effective amount of the nucleic acid molecule, the plasmid, the rAAV, or the composition to the subject.

Embodiment 36. The method of any one of claims 32-35, wherein the cell is an ear cell.

Embodiment 37. The method of claim 36, wherein the cell is an inner hair cell (IHC), or an outer hair cell (OHC).

Embodiment 38. The method of any one of claims 32-36, wherein the cell is a vestibular hair cell, spiral ganglion, or vestibular ganglion.

Embodiment 39. A method of treating a genetic hearing loss in a subject in need thereof, comprising: administering to the ear of the subject for an administration period, a therapeutically effective amount of the nucleic acid molecule of any one of claims 1-17, the plasmid of claim 18, the cell of claim 19, or the rAAV of any one of claims 21-30, or the composition of claim 31, thereby treating the genetic hearing loss in the subject.

Embodiment 40. A method of treating a genetic hearing loss in a subject in need thereof, comprising: administering to the ear of the subject for an administration period, a therapeutically effective amount of the rAAV of claim 26, thereby treating the genetic hearing loss in the subject.

Embodiment 41. The method of claim 39 or claim 40, wherein the subject suffers from, or is at a risk of developing the genetic hearing loss.

Embodiment 42. The method of any one of claims 39-41, wherein the genetic hearing loss is an autosomal recessive non-syndromic hearing loss (ARNSHL).

Embodiment 43. The method of any one of claims 39-42, wherein the genetic hearing loss is associated with, promoted by, or caused by a mutation in the transmembrane channel-like 1 (TMC1)-encoding gene.

Embodiment 44. The method of claim 43, wherein the mutation in the TMC1-encoding gene is present at the DFNB7/11 locus on chromosome 9q31-21.

Embodiment 45. The method of any one of claims 39-44, wherein the method comprises diminishing the severity of; delaying the onset or progression of; and/or eliminating a symptom of the genetic hearing loss.

Embodiment 46. The method of claim 45, wherein symptom of the genetic hearing loss comprises: a reduced ability to hear, or an inability to hear.

Embodiment 47. The method of any one of claims 39-46, wherein the genetic hearing loss is: (a) a progressive genetic hearing loss, (b) a pre-lingual genetic hearing loss, (c) a congenital genetic hearing loss, or (d) any combination thereof.

Embodiment 48. The method of any one of claims 39-47, wherein the subject has a hearing threshold in the range of about 25 dB to about 80 dB.

Embodiment 49. The method of claim 48, wherein the subject has a hearing threshold of about 40 dB.

Embodiment 50. The method of claim 48, wherein the subject has a hearing threshold of about 55 dB.

Embodiment 51. The method of claim 48, wherein the subject has a hearing threshold of about 70 dB.

Embodiment 52. The method of any one of claims 39-51, wherein the method comprises decreasing the hearing threshold of the subject during or after the administration period, as compared to prior to the administration period.

Embodiment 53. The method of any one of claims 39-52, wherein, the hearing threshold of the subject during or after the administration period is lower, as compared to before the administration period.

Embodiment 54. The method of any one of claims 39-53, wherein, the hearing threshold of the subject during or after the administration period is at least 5% lower, as compared to before the administration period.

Embodiment 55. The method of any one of claims 39-54, wherein the hearing threshold of the subject during or after the administration period is lower, as compared to that of a control subject, wherein the control subject is administered an rAAV, comprising: an AAV9-php.b capsid protein; and a nucleic acid molecule, comprising an AAV expression cassette, wherein the AAV expression cassette lacks a woodchuck hepatitis virus posttranscriptional regulatory element (WPRE) and comprises, from 5′ to 3′: a 5′ AAV ITR, a CB6 promoter, a Kozak sequence, a transgene, encoding a codon-optimized human transmembrane channel-like 1 (co-hTMC1), a beta globin polyadenylation sequence, and a 3′ AAV ITR.

Embodiment 56. The method of any one of claims 39-55, wherein the hearing threshold of the subject during or after the administration period is lower for a longer period of time, as compared to a control subject, wherein the control subject is administered an rAAV, comprising: an AAV9-php.b capsid protein; and a nucleic acid molecule, comprising an AAV expression cassette, wherein the AAV expression cassette lacks a woodchuck hepatitis virus posttranscriptional regulatory element (WPRE) and comprises, from 5′ to 3′: a 5′ AAV ITR, a CB6 promoter, a Kozak sequence, a transgene, encoding a codon-optimized human transmembrane channel-like 1 (co-hTMC1), a beta globin polyadenylation sequence, and a 3′ AAV ITR.

Embodiment 57. The method of any one of claims 39-56, wherein the hearing threshold of the subject during or after the administration period is lower, as compared to that of a control subject, wherein the control subject is administered an rAAV, comprising: an AAV9-php.b capsid protein; and a nucleic acid molecule, comprising an AAV expression cassette, wherein the AAV expression cassette lacks a CB6 promoter and comprises, from 5′ to 3′: a 5′ AAV ITR, a CMV promoter, a Kozak sequence, a transgene, encoding a codon-optimized human transmembrane channel-like 1 (co-hTMC1), a beta globin polyadenylation sequence, a woodchuck hepatitis virus posttranscriptional regulatory element (WPRE) and a 3′ AAV ITR.

Embodiment 58. The method of any one of claims 39-57, wherein the hearing threshold of the subject during or after the administration period is lower for a longer period of time, as compared to that of a control subject, wherein the control subject is administered an rAAV, comprising: an AAV9-php.b capsid protein; and a nucleic acid molecule, comprising an AAV expression cassette, wherein the AAV expression cassette lacks a CB6 promoter and comprises, from 5′ to 3′: a 5′ AAV ITR, a CMV promoter, a Kozak sequence, a transgene, encoding a codon-optimized human transmembrane channel-like 1 (co-hTMC1), a beta globin polyadenylation sequence, a woodchuck hepatitis virus posttranscriptional regulatory element (WPRE) and a 3′ AAV ITR.

Embodiment 59. The method of any one of claims 39-58, wherein the survival of inner hair cells and/or outer hair cells in the ear of the subject during or after the administration period is improved as compared to the survival of inner hair cells and/or outer hair cells in the ear prior to the administration period.

Embodiment 60. The method of any one of claims 39-59, wherein the number of inner hair cells and/or outer hair cells in the ear of the subject during or after the administration period is higher, as compared to the number of inner hair cells and/or outer hair cells in the ear before the administration period.

Embodiment 61. The method of any one of claims 39-60, wherein the nucleic acid molecule, the plasmid, the cell, the rAAV, or the composition is administered via intracochlear delivery.

Embodiment 62. The method of any one of claims 39-61, wherein the subject is a human subject.

Embodiment 63. The method of claim 62, wherein the subject is a neonate or an infant.

Embodiment 64. The method of claim 62, wherein the subject is less than 18 years of age.

Embodiment 65. The method of claim 62, wherein the subject is at least 18 years of age.

Embodiment 66. The method of any one of claims 62-65, wherein the subject has autosomal dominant non-syndromic sensorineural hearing loss.

Embodiment 67. The method of any one of claims 62-65, wherein the subject has autosomal recessive non-syndromic neurosensory deafness.

What is claimed is:

1. A nucleic acid molecule, comprising an adeno-associated virus (AAV) expression cassette, wherein the AAV expression cassette comprises, from 5′ to 3′:
a 5′ AAV inverted terminal repeat (ITR);
a promoter comprising a CB6 promoter;
a transgene encoding transmembrane channel-like 1 (TMC1); and
a 3′ AAV ITR.

2. The nucleic acid molecule of claim 1, wherein the transgene encodes a human transmembrane channel-like 1 (hTMC1).

3. The nucleic acid molecule of claim 1 or claim 2, wherein the transgene encodes a codon-optimized human transmembrane channel-like 1 (co-hTMC1).

4. The nucleic acid molecule of any one of claims 1-3, wherein the transgene comprises a nucleic acid sequence having at least 90% identity to SEQ ID NO: 1.

5. The nucleic acid molecule of any one of claims 1-4, wherein the transgene comprises the nucleic acid sequence of SEQ ID NO: 1.

6. The nucleic acid molecule of any one of claims 1-5, wherein the promoter is operably linked to the transgene.

7. The nucleic acid molecule of any one of claims 1-6, wherein the CB6 promoter comprises the nucleic acid sequence of SEQ ID NO: 2.

8. The nucleic acid molecule of any one of claims 1-7 wherein the AAV expression cassette comprises a beta globin polyadenylation sequence.

9. The nucleic acid molecule of claim 8, wherein the beta globin polyadenylation sequence comprises the nucleic acid sequence of SEQ ID NO: 3.

10. The nucleic acid molecule of any one of claims 1-9, wherein the AAV expression cassette comprises a Kozak sequence.

11. The nucleic acid molecule of claim 10, wherein the Kozak sequence comprises the nucleic acid sequence of SEQ ID NO: 4.

12. The nucleic acid molecule of any one of claims 1-11, wherein the AAV expression cassette comprises a woodchuck hepatitis virus posttranscriptional regulatory element (WPRE).

13. The nucleic acid molecule of claim 12, wherein the WPRE comprises the nucleic acid sequence of SEQ ID NO: 5.

14. The nucleic acid molecule of any one of claims 1-13, wherein the 5′ AAV ITR sequence comprises the nucleic acid sequence of SEQ ID NO: 6.

15. The nucleic acid molecule of any one of claims 1-14, wherein the 3′ AAV ITR sequence comprises the nucleic acid sequence of SEQ ID NO: 7.

16. The nucleic acid molecule of any one of claims 1-15, wherein the AAV expression cassette comprises, from 5′ to 3′:
a 5′ AAV ITR,
a CB6 promoter,
a Kozak sequence,
a codon-optimized human transmembrane channel-like 1 (co-hTMC1),
a woodchuck hepatitis virus posttranscriptional regulatory element (WPRE),
a beta globin polyadenylation sequence, and
a 3′ AAV ITR.

17. The nucleic acid molecule of any one of claims 1-16, wherein the AAV expression cassette comprises the nucleic acid sequence of SEQ ID NO: 8.

18. A plasmid, comprising the nucleic acid molecule of any one of claims 1-17.

19. A cell, comprising the nucleic acid molecule of any one of claims 1-17, or the plasmid of claim 18.

20. A method of producing a recombinant adeno-associated virus (rAAV), the method comprising:
contacting an AAV producer cell with the nucleic acid molecule of any one of claims 1-17, or the plasmid of claim 18.

21. A recombinant adeno-associated virus (rAAV) produced by the method of claim 20.

22. The rAAV of claim 21, wherein the rAAV comprises an AAV9-php.b capsid protein.

23. The rAAV of claim 21, wherein the rAAV comprises a AAV9 capsid protein, comprising an insertion of the amino acid sequence of SEQ ID NO: 11 (TLAVPFK) between amino acid 588 and amino acid 589, wherein the amino acids are numbered according to VP1 capsid protein.

24. A recombinant adeno-associated virus (rAAV), comprising:
an AAV9-php.b capsid protein; and the nucleic acid molecule of any one of claims 1-17.

25. A recombinant adeno-associated virus (rAAV), comprising: an AAV9-php.b capsid protein; and a nucleic acid molecule comprising an AAV expression cassette, wherein the AAV expression cassette comprises, from 5′ to 3′:
a 5′ AAV ITR comprising the nucleic acid sequence of SEQ ID NO: 6,
a CB6 promoter comprising the nucleic acid sequence of SEQ ID NO: 2,
a Kozak sequence comprising the nucleic acid sequence of SEQ ID NO: 4,
a transgene, encoding a codon-optimized human transmembrane channel-like 1 (co-hTMC1), comprising the nucleic acid sequence of SEQ ID NO: 1,
a woodchuck hepatitis virus posttranscriptional regulatory element (WPRE) comprising the nucleic acid sequence of SEQ ID NO: 5,
a beta globin polyadenylation sequence comprising the nucleic acid sequence of SEQ ID NO: 3, and
a 3′ AAV ITR comprising the nucleic acid sequence of SEQ ID NO: 7.

26. A recombinant adeno-associated virus (rAAV), comprising:
an AAV9-php.b capsid protein; and
a nucleic acid molecule, comprising an AAV expression cassette, wherein the AAV expression cassette comprises the nucleic acid sequence of SEQ ID NO: 8.

27. The rAAV of any one of claims 22-26, wherein the AAV9.php.b capsid protein is encoded by a nucleic acid having at least 90% identity to SEQ ID NO: 13.

28. The rAAV of any one of claims 22-27, wherein the AAV expression cassette comprises an miR-1 binding site, an miR-133a binding site, and/or an miR-122 binding site.

29. The rAAV of any one of claims 22-28, wherein the rAAV is a self-complementary AAV.

30. The rAAV of any one of claims 22-28, wherein the rAAV is a single-stranded AAV.

31. A pharmaceutical composition, comprising:
(a) the nucleic acid molecule of any one of claims 1-17, the plasmid of claim 18, the cell of claim 19, or the rAAV of any one of claims 21-30; and
(b) a pharmaceutically acceptable carrier.

32. A method of expressing transmembrane channel-like 1 (TMC1) in a cell, comprising:
contacting the cell with the nucleic acid molecule of any one of claims 1-17, the plasmid of claim 18, the cell of claim 19, or the rAAV of any one of claims 21-30, or the composition of claim 31, thereby expressing TMC1 in the cell.

33. The method of claim 32, wherein the contacting step is performed in vitro, ex vivo, or in vivo.

34. The method of claim 33, wherein the contacting step is performed in vivo in a subject in need thereof.

35. The method of claim 34, wherein the contacting step comprises administering a therapeutically effective amount of the nucleic acid molecule, the plasmid, the rAAV, or the composition to the subject.

36. The method of any one of claims 32-35, wherein the cell is an ear cell.

37. The method of claim 36, wherein the cell is an inner hair cell (IHC), or an outer hair cell (OHC).

38. The method of any one of claims 32-36, wherein the cell is a vestibular hair cell, spiral ganglion, or vestibular ganglion.

39. A method of treating a genetic hearing loss in a subject in need thereof, comprising: administering to the ear of the subject for an administration period, a therapeutically effective amount of the nucleic acid molecule of any one of claims 1-17, the plasmid of claim 18, the cell of claim 19, or the rAAV of any one of claims 21-30, or the composition of claim 31, thereby treating the genetic hearing loss in the subject.

40. A method of treating a genetic hearing loss in a subject in need thereof, comprising: administering to the ear of the subject for an administration period, a therapeutically effective amount of the rAAV of claim 26, thereby treating the genetic hearing loss in the subject.

41. The method of claim 39 or claim 40, wherein the subject suffers from, or is at a risk of developing the genetic hearing loss.

42. The method of any one of claims 39-41, wherein the genetic hearing loss is an autosomal recessive non-syndromic hearing loss (ARNSHL).

43. The method of any one of claims 39-42, wherein the genetic hearing loss is associated with, promoted by, or caused by a mutation in the transmembrane channel-like 1 (TMC1)-encoding gene.

44. The method of claim 43, wherein the mutation in the TMC1-encoding gene is present at the DFNB7/11 locus on chromosome 9q31-21.

45. The method of any one of claims 39-44, wherein the method comprises diminishing the severity of, delaying the onset or progression of; and/or eliminating a symptom of the genetic hearing loss.

46. The method of claim 45, wherein symptom of the genetic hearing loss comprises: a reduced ability to hear, or an inability to hear.

47. The method of any one of claims 39-46, wherein the genetic hearing loss is: (a) a progressive genetic hearing loss, (b) a pre-lingual genetic hearing loss, (c) a congenital genetic hearing loss, or (d) any combination thereof.

48. The method of any one of claims 39-47, wherein the subject has a hearing threshold in the range of about 25 dB to about 80 dB.

49. The method of claim 48, wherein the subject has a hearing threshold of about 40 dB.

50. The method of claim 48, wherein the subject has a hearing threshold of about 55 dB.

51. The method of claim 48, wherein the subject has a hearing threshold of about 70 dB.

52. The method of any one of claims 39-51, wherein the method comprises decreasing the hearing threshold of the subject during or after the administration period, as compared to prior to the administration period.

53. The method of any one of claims 39-52, wherein, the hearing threshold of the subject during or after the administration period is lower, as compared to before the administration period.

54. The method of any one of claims 39-53, wherein, the hearing threshold of the subject during or after the administration period is at least 5% lower, as compared to before the administration period.

55. The method of any one of claims 39-54, wherein the hearing threshold of the subject during or after the administration period is lower, as compared to that of a control subject, wherein the control subject is administered an rAAV, comprising: an AAV9-php.b capsid protein; and a nucleic acid molecule, comprising an AAV expression cassette, wherein the AAV expression cassette lacks a woodchuck hepatitis virus posttranscriptional regulatory element (WPRE) and comprises, from 5′ to 3′: a 5′ AAV ITR, a CB6 promoter, a Kozak sequence, a transgene, encoding a codon-optimized human transmembrane channel-like 1 (co-hTMC1), a beta globin polyadenylation sequence, and a 3′ AAV ITR.

56. The method of any one of claims 39-55, wherein the hearing threshold of the subject during or after the administration period is lower for a longer period of time, as compared to a control subject, wherein the control subject is administered an rAAV, comprising: an AAV9-php.b capsid protein; and a nucleic acid molecule, comprising an AAV expression cassette, wherein the AAV expression cassette lacks a woodchuck hepatitis virus posttranscriptional regulatory element (WPRE) and comprises, from 5′ to 3′: a 5′ AAV ITR, a CB6 promoter, a Kozak sequence, a transgene, encoding a codon-optimized human transmembrane channel-like 1 (co-hTMC1), a beta globin polyadenylation sequence, and a 3′ AAV ITR.

57. The method of any one of claims 39-56, wherein the hearing threshold of the subject during or after the administration period is lower, as compared to that of a control subject, wherein the control subject is administered an rAAV, comprising: an AAV9-php.b capsid protein; and a nucleic acid molecule, comprising an AAV expression cassette, wherein the AAV expression cassette lacks a CB6 promoter and comprises, from 5′ to 3′: a 5′ AAV ITR, a CMV promoter, a Kozak sequence, a transgene, encoding a codon-optimized human transmembrane channel-like 1 (co-hTMC1), a beta globin polyadenylation sequence, a woodchuck hepatitis virus posttranscriptional regulatory element (WPRE) and a 3′ AAV ITR.

58. The method of any one of claims 39-57, wherein the hearing threshold of the subject during or after the administration period is lower for a longer period of time, as compared to that of a control subject, wherein the control subject is administered an rAAV, comprising: an AAV9-php.b capsid protein; and a nucleic acid molecule, comprising an AAV expression cassette, wherein the AAV expression cassette lacks a CB6 promoter and comprises, from 5′ to 3′: a 5′ AAV ITR, a CMV promoter, a Kozak sequence, a transgene, encoding a codon-optimized human transmembrane channel-like 1 (co-hTMC1), a beta globin polyadenylation sequence, a woodchuck hepatitis virus posttranscriptional regulatory element (WPRE) and a 3′ AAV ITR.

59. The method of any one of claims 39-58, wherein the survival of inner hair cells and/or outer hair cells in the ear of the subject during or after the administration period is improved as compared to the survival of inner hair cells and/or outer hair cells in the ear prior to the administration period.

60. The method of any one of claims 39-59, wherein the number of inner hair cells and/or outer hair cells in the ear of the subject during or after the administration period is higher, as compared to the number of inner hair cells and/or outer hair cells in the ear before the administration period.

61. The method of any one of claims 39-60, wherein the nucleic acid molecule, the plasmid, the cell, the rAAV, or the composition is administered via intracochlear delivery.

62. The method of any one of claims 39-61, wherein the subject is a human subject.

63. The method of claim 62, wherein the subject is a neonate or an infant.

64. The method of claim 62, wherein the subject is less than 18 years of age.

65. The method of claim 62, wherein the subject is at least 18 years of age.

66. The method of any one of claims 62-65, wherein the subject has autosomal dominant non-syndromic sensorineural hearing loss.

67. The method of any one of claims 62-65, wherein the subject has autosomal recessive non-syndromic neurosensory deafness.
